SEQUENCE LISTING (9 sequences)

SEQ ID NO: 1 - DNA, 528 bp, Artificial sequence
  Synthetic oligonucleotide
atgggggctccggcaaccaggcgctgcgtggagtggctgctgggcctctacttcctcagccacatccccatcaccctgttcatggacctgcaggcggtgctgccgcgcgagctctacccagtcgagtttagaaacctgctgaagtggtatgctaaggagttcaaagacccactgctacaggagcccccagcctggtttaagtcctttctgttttgcgagcttgtgtttcagctgcctttctttcccattgcaacgtatgccttcctcaaaggaagctgcaagtggattcgaactcctgcaatcatctactctgttcacaccatgacaaccttaattccgatactctccacatttctgtttgaggatttctccaaagccagtggtttcaagggacaaagacctgagactttgcatgaacggttaacccttgtgtctgtctatgccccctacttactcatcccattcatacttttaattttcatgttgcggagcccctactacaagtatgaagagaaaagaaaaaaaaaa

SEQ ID NO: 2 - protein, 176 aa, Artificial sequence
  Synthetic polypeptide
MGAPATRRCVEWLLGLYFLSHIPITLFMDLQAVLPRELYPVEFRNLLKWYAKEFKDPLLQEPPAWFKSFLFCELVFQLPFFPIATYAFLKGSCKWIRTPAIIYSVHTMTTLIPILSTFLFEDFSKASGFKGQRPETLHERLTLVSVYAPYLLIPFILLIFMLRSPYYKYEEKRKKK

SEQ ID NO: 3 - protein, 19 aa, Artificial sequence
  Synthetic peptide
ATNFSLLKQAGDVEENPGP

SEQ ID NO: 4 - RNA, 25 nt, Artificial sequence
  Synthetic oligonucleotide
caaccuguugcggugguacucuaag

SEQ ID NO: 5 - RNA, 25 nt, Artificial sequence
  Synthetic oligonucleotide
cuuagaguaccaccgcaacagguug

SEQ ID NO: 6 - DNA, 20 bp, Artificial sequence
  Synthetic primer
tacttcgtctcgcacatccc

SEQ ID NO: 7 - DNA, 20 bp, Artificial sequence
  Synthetic primer
ttgctgaactcctgcgggta

SEQ ID NO: 8 - DNA, 21 bp, Artificial sequence
  Synthetic primer
cccgcgagtacaaccttcttg

SEQ ID NO: 9 - DNA, 21 bp, Artificial sequence
  Synthetic primer
gtcatccatggcgaactggtg

(19) United States
(12) Patent Application Publication
(10) Pub. No.: US 2020/0000822 A1
(43) Pub. Date: Jan. 2, 2020

(54) METHODS OF TREATMENT FOR CANCER, STEROL HOMEOSTASIS, AND NEUROLOGICAL DISEASES

(71) Applicants: BOARD OF REGENTS, THE UNIVERSITY OF TEXAS SYSTEM, Austin, TX (US); PRESIDENT AND FELLOWS OF HARVARD COLLEGE, Cambridge, MA (US)

(72) Inventors: Andrew Curtis KRUSE, Boston, MA (US); Assaf ALON, Boston, MA (US); Hayden R. SCHMIDT, Boston, MA (US); Stephen MARTIN, Austin, TX (US); James SAHN, Austin, TX (US); Michael WOOD, Austin, TX (US)

(21) Appl. No.: 16/488,010

(22) PCT Filed: Feb. 23, 2018
(86) PCT No.: PCT/US2018/019403

§ 371 Date: Aug. 22, 2019

Related U.S. Application Data
(60) Provisional application No. 62/462,435, filed on Feb. 23, 2017.

(51) Int. Cl. A61K 31/55; A61K 31/454; A61K 31/404; A61K 31/438; A61K 31/496; A61K 31/5377; A61K 31/445; A61K 31/473; C07K 16/28; C12N 15/113

(52) U.S. Cl. CPC A61K 31/55; A61K 31/454; A61K 31/404; A61K 31/438; A61K 31/496; C12N 2310/14; A61K 31/445; A61K 31/473; C07K 16/2869; C12N 15/1138; A61K 31/5377

(57) ABSTRACT

The present disclosure provides methods of treating cancer, sterol homeostasis diseases, and neurological diseases using compounds which modulate the activity of sigma receptors. In particular, the present disclosure provides method s of modulating the sigma 2 receptor for use in treating one or more diseases associated with that sigma receptor.

This application claims the benefit of priority to U.S. Provisional Application No. 62/462,435, filed on Feb. 23, 2017, the entire contents of which are incorporated herein by reference.

### BACKGROUND OF THE INVENTION

The sigma-2 receptor is a poorly understood transmembrane protein implicated in diseases as diverse as cancer, Alzheimer's disease, and schizophrenia. Unlike virtually all other pharmacologically defined receptors, the sigma-2 receptor has eluded molecular cloning since its discovery. However, a number of ligands have been discovered that treat sigma-2 associated diseases. TMEM97, a four-pass ER-resident transmembrane protein has recently been shown to be a binding partner of NPC1, a lysosomal cholesterol transporter. Loss of NPC1 function causes Niemann-Pick Disease type C1, an autosomal recessive disorder with no known effective treatments. Knockdown of TMEM97 by RNA interference has been shown to ameliorate the cellular effects of NPC1 disease-causing mutations, offering an attractive platform on which to base subsequent therapeutic strategies.

### SUMMARY OF THE INVENTION

This invention identifies the long elusive sigma-2 receptor as TMEM97. The cloning of the sigma-2 receptor resolves a longstanding pharmacological mystery and unites an emerging drug target with a number of established therapeutic molecules. Methods of treatment for cancer, sterol homeostasis diseases, and neurological diseases are disclosed herein.

In one aspect, this invention features a method of treating a subject having a sterol homeostasis disease, the method comprising administering to the subject a sigma-2 receptor ligand in an amount and for a duration sufficient to treat the sterol homeostasis disease.

In some embodiments, the ligand is a sigma-2 receptor agonist, antagonist, or partial agonist.

In some embodiments, the ligand is selected from the group consisting of: opipramol, MIN-101 (2-[[1-[2-(4-fluorophenyl)-2-oxoethyl]piperidin-4-yl]methyl]-3H-isoindol-1-one), CT-1812, siramesine, rimcazole, ibogaine, afobazole, BMY-14802 (1-(4-Fluorophenyl)-4-[4-(5-fluoro-2-pyrimidinyl)-1-piperazinyl]-1-butanol), and panamesine.

In some embodiments, the ligand is selected from the group consisting of: ¹¹C-PB-28, ¹²⁵I RHM-4, ¹²⁵I-IAC44, ¹²⁵I-IAF(1-N-(2′,6′-dimethyl-morpholino)-3-(4-azido-3-[(125)I]iodo-phenyl)propane, ¹⁸F ISO-1, 2-(4-(3-(4-fluorophenyl)indol-1-yl)butyl)-6,7-dimethoxy-1,2,3,4-tetrahydroisoquinoline), ³H DTG, ³H-azido-DTG, ³H-PB28, ³H-RHM-1, ^{99m}Tc BAT-EN6, ^{99m}Tc-4-(4-cyclohexylpiperazine-1-yl)-butan-1-one-1-cyclopentadienyltricarbonyl technetium, ABN-1, AG-205, ANSTO-19, benzoxazolone, BIMU-1, CB-182, CB-184, CB-64D, CB-64L, cocaine, ditolylguanidine (DTG), F281, indole ((1-[3-[4-(substituted-phenyl) piperazin-1-yl]-propyl]-1H-indole, K05-138, K05-138, N-Benzyl-7-azabicyclo[2.2.1]heptane, PB183, PB28, RHM-1, RHM-138, RHM-2, RHM-4, SM-21, SN79, SV119, SW107, SW116, SW120, SW43, TC4ANSTO-19, WC-21, WC-26, WC-59, yun179, yun194, yun201, yun202, yun203, yun204, yun209, yun210, yun212, yun234, yun236, yun242, yun243 (RMH-1), yun245, yun250, yun251, yun253, yun254, yun552, SAS-0132, DKR-1051, DKR-1005, JVW-1009, and SAS-1121.

In some embodiments, the ligand is a compound having the formula:

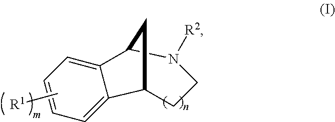

wherein:
R¹ is hydrogen, halogen (e.g., —Cl), —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —N₃, —CF₃, —CCl₃, —CBr₃, —CCI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙNR⁴R^{4A}, —NHNR⁴R^{4A}, —ONR⁴R^{4A}, —NHC(O)NHNR⁴R^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n1 and n2 are independently 1 or 2;
m is 1, 2, 3 or 4;
n is 1 or 2; and
R³, R^{3A}, R⁴, R^{4A} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
In embodiments, R² is hydrogen, —CF₃, —CN, —C(O)R⁴, —OR⁴, —NR⁴R^{4A}, —C(O)OR⁴, —C(O)NR⁴R^{4A}, —NO₂, —S(O)ₙ₂R⁴, —S(O)ₙ₂OR⁴, —S(O)ₙ₂NR⁴R^{4A}, —NHNR⁴R^{4A}, —NHC(O)NHNR⁴R^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, the ligand is a compound having the formula:

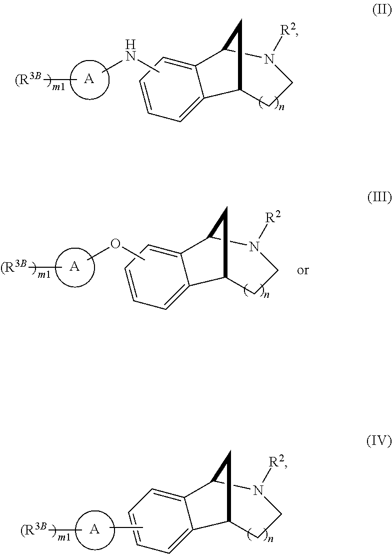

wherein
R^{3B} is —CF₃, —CN, —OH, —NH₂, —CONH₂, —S(O)₃H, —S(O)₂NH₂, —NHC(O) NH₂, —NHC(O)H, —OCHF₂, oxo, halogen, —COOH, —NO₂, —SH, —S(O)₄H, —NHNH₂, —ONH₂, —NHC(O)NHNH₂, —NHS(O)₂H, —NHC(O)—OH, —NHOH, —OCF₃, unsubstituted alkyl, unsubstituted heteroalkyl, unsubstituted cycloalkyl, unsubstituted heterocycloalkyl, unsubstituted aryl or unsubstituted heteroaryl;
ring A is aryl, heteroaryl, cycloalkyl or heterocycloalkyl; and
m1 is 0, 1, 2, 3, or 4.

In some embodiments, the ligand is a compound having the formula:

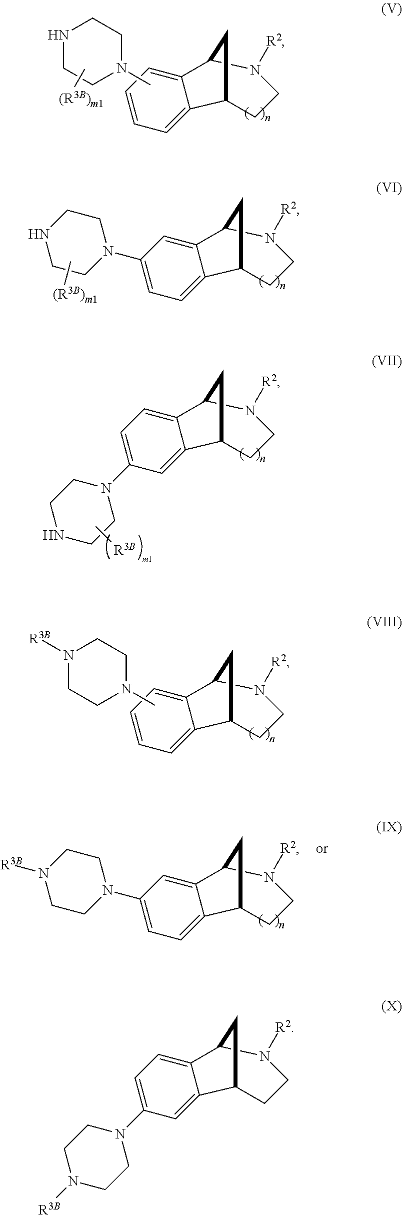

In some embodiments, the ligand is a compound having the formula:

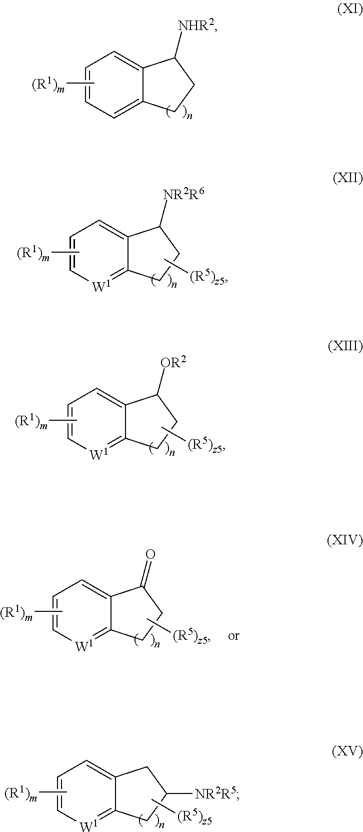

wherein R¹ is hydrogen, halogen (e.g., —F, —Cl, —Br, —I), —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, substituted or unsubstituted alkyl, substituted or unsubstituted heteroalkyl, substituted or unsubstituted cycloalkyl, substituted or unsubstituted heterocycloalkyl (e.g., piperazinyl, piperidinyl, morpholinyl), substituted or unsubstituted aryl (e.g., phenyl), or substituted or unsubstituted heteroaryl (e.g., pyridyl); R² is hydrogen, halogen, —N₃, —CF₃, —CCl₃, —CBr₃, —CI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, substituted or unsubstituted alkyl (e.g., —CH₃, —CH₂CH₃, —CH₂CH₂CH₃, —CH₂CH₂CH₂OH, —CH₂Ph), substituted or unsubstituted heteroalkyl (e.g., —C(O)OCH₂Ph, —C(O)NHCH₂Ph, —CH₂CH₂C(O)OCH₂CH₃, —CH₂CH₂C(O)OCH₃, —CH₂CH₂OCH₂CH₃, —CH₂CH₂OCH₃), substituted or unsubstituted cycloalkyl, substituted or unsubstituted heterocycloalkyl (e.g., tetrahydropyranyl, piperidinyl, methyl substituted piperidinyl), substituted or unsubstituted aryl, or substituted or unsubstituted heteroaryl; the symbols n1 and n2 are independently 1 or 2; the symbol m is 1, 2, 3 or 4; n is 1, 2, 3 or 4; R³, R^{3A}, R⁴, R^{4A} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is halogen, —N₃, —CF₃, —CCl₃, —CBr₃, —CI₃, —CN, —C(O)R^{5C}, —OR^{5D} (e.g., —OH), —NR^{5A}R^{5B}, —C(O)OR^{5D}, —C(O)NR^{5A}R^{5B}, —NO₂, —SR^{5D}, —S(O)ₙ₅R^{5C}, —S(O)ₙ₅OR^{5D}, —S(O)ₙ₅NR^{5A}R^{5B}, —NHNR^{5A}R^{5B}, —ONR^{5A}R^{5B}, —NHC(O)NHNR^{5A}R^{5B}, substituted or unsubstituted alkyl (e.g., —CH₂Ph), substituted or unsubstituted heteroalkyl, substituted or unsubstituted cycloalkyl, substituted or unsubstituted heterocycloalkyl, substituted or unsubstituted aryl, or substituted or unsubstituted heteroaryl; the symbol n5 is independently 1 or 2; the symbol z5 is independently an integer from 0 to 6; R⁶ is halogen, —N₃, —CF₃, —CCl₃, —CBr₃, —CI₃, —CN, —C(O)R^{6C}, —OR^{6D}, —NR^{6A}R^{6B}, —C(O)OR^{6D}, —C(O)NR^{6A}R^{6B}, —NO₂, —SR^{6D}, —S(O)ₙ₆R^{6C}, —S(O)ₙ₆OR^{6D}, —S(O)ₙ₆NR^{6A}R^{6B}, —NHNR^{6A}R^{6B}, —ONR^{6A}R^{6B}, —NHC(O)NHNR^{6A}R^{6B}, substituted or unsubstituted alkyl (e.g., —CH₃, —CH₂CH₃, —CH₂CH₂CH₃, —CH₂CH₂CH₂OH, —CH₂Ph), substituted or unsubstituted heteroalkyl (e.g., —C(O)OCH₂Ph, —CH₂CH₂C(O)OCH₂CH₃, —CH₂CH₂C(O)OCH₃, —CH₂CH₂OCH₂CH₃, —CH₂CH₂OCH₃), substituted or unsubstituted cycloalkyl, substituted or unsubstituted heterocycloalkyl (e.g., tetrahydropyranyl, piperidinyl, methyl substituted piperidinyl), substituted or unsubstituted aryl, or substituted or unsubstituted heteroaryl; the symbol n6 is independently 1 or 2; W¹ is CH, C(R¹), or N; and R^{5A}, R^{5B}, R^{5C}, R^{5D}, R^{6A}, R^{6B}, R^{6C} and R^{6D} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² is hydrogen, —CF₃, —CN, —C(O)R⁴, —OR⁴, —NR⁴R^{4A}, —C(O)OR⁴, —C(O)NR⁴R^{4A}, —NO₂, —S(O)ₙ₂R⁴, —S(O)ₙ₂OR⁴, —S(O)ₙ₂NR⁴R^{4A}, —NHNR⁴R^{4A}, —NHC(O)NHNR⁴R^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, the ligand is a compound having the formula:

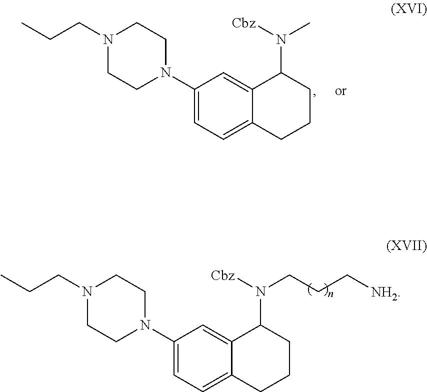

In some embodiments, the sterol homeostasis disease is Niemann-Pick disease, such as Niemann-Pick type C disease, or Niemann-Pick type C1 disease.

In another aspect, this invention features a method of treating a subject having a neurological condition, the method comprising administering to the subject a TMEM97 ligand in an amount and for a duration sufficient to treat the neurological condition.

In some embodiments, the ligand is a TMEM97 agonist, antagonist, or partial agonist.

In some embodiments, the ligand is Elacridar or Ro 48-8071 (4-Bromophenyl)-[2-fluoro-4-[6-[methyl(prop-2-enyl)amino]hexoxy]phenyl]methanone).

In some embodiments, the ligand is an anti-TMEM97 antibody.

In another aspect, this invention features a method of treating a subject having a neurological condition, the method comprising administering to the subject a microRNA, siRNA, or antisense RNA that targets TMEM97 expression in an amount and for a duration sufficient to treat the neurological condition.

In some embodiments, the neurological condition is selected from a group consisting of: conditions requiring neuroprotection, stroke, anxiety, depression, Alzheimer's disease, frontotemporal dementia, Lewy Body dementia, Pick's disease, Huntington's disease, pain, Parkinson's disease, multiple sclerosis, microglia inflammation, schizophrenia, addiction, and head injury (e.g., concussion or traumatic brain injury). In some embodiments, the neurological condition is selected from a group consisting of: conditions requiring neuroprotection, stroke, anxiety, depression, Alzheimer's disease, frontotemporal dementia, Lewy Body dementia, Pick's disease, Huntington's disease, Parkinson's disease, multiple sclerosis, microglia inflammation, schizophrenia, and head injury (e.g., concussion or traumatic brain injury).

In other embodiments, the neurological condition is pain, including neuropathic pain, and addiction, including opiod, cocaine, methamphetamine, and alcohol. In other embodiments, the neurological condition is pain (e.g., neuropathic pain). In other embodiments, the neurological condition is addiction (e.g., addiction to a drug or chemical agent, addition to an opioid, cocaine, methamphetamine, or alcohol).

In another aspect, this invention features a method of treating cancer, the method comprising administering to the subject a sigma-2 receptor ligand or TMEM97 ligand in an amount and for a duration sufficient to treat the cancer.

In some embodiments, the cancer is squamous cell carcinoma, glioma, colorectal cancer, gastric cancer, epithelial ovarian cancer, ovarian cancer, pancreatic cancer, melanoma; non-small-cell lung cancer, or breast cancer (e.g., triple negative breast cancer), or a multi drug resistant (MDR) variety of any of the foregoing (e.g., MDR-ovarian cancer).

In some embodiments, the sigma-2 receptor ligand or TMEM97 ligand is selected from the group consisting of: compounds of formula I, II, III, IV, V, VI, VII, VIII, IX, X, XI, XII, XIII, XIV, XV, XVI, and XVII.

### Definitions

As used herein, a “sterol homeostasis disease” is a disease in which the normal equilibrium of natural steroid alcohols is disrupted. Some examples include Niemann-Pick disease, and Smith-Lemli-Opitz syndrome (SLOS).

As used herein, a “sigma-2 receptor ligand” is any ligand that selectively or specifically binds to the Sigma-2 receptor, specifically excluding ligands previously identified to bind TMEM97 (e.g., Elacridar and Ro 48-8071).

As used herein, a ligand that “specifically binds” is one that binds its receptor with an affinity of at least 10⁻⁶ M (e.g., at least 10⁻⁷ M, 10⁻⁸ M, 10⁻⁹ M, 10⁻¹⁰ M, or 10⁻¹¹ M.) In certain embodiments, a ligand that specifically binds is one that binds its receptor with at least five-fold greater affinity as compared to any non-targets, e.g., at least 10-, 20-, 50-, or 100-fold greater affinity. A ligand can be an agonist, antagonist, or partial agonist.

As used herein, an “agonist” is defined as a ligand that has at least 90% of cytotoxic activity of siramesine in a tumor cell apoptosis assay with EMT-6 or human melanoma cell line MDA-MB-435.

As used herein, an “antagonist” is defined as a ligand that has less than 10% of cytotoxic activity of siramesine in a tumor cell apoptosis assay with EMT-6 and human melanoma cell line MDA-MB-435.

As used herein, a “partial agonist” is defined as a ligand that has between 10% and 90% of cytotoxic activity of siramesine in a tumor cell apoptosis assay with EMT-6 and human melanoma cell line MDA-MB-435.

As used herein, a “Niemann-Pick disease” is a metabolic disorder in which sphingomyelin accumulates in cell lysosomes due to dysfunctional metabolism of sphingolipids. Niemann-Pick disease types A and B are associated with mutations in the SMPD1 gene while Niemann-Pick disease type C is associated with mutations in the NPC1 (type C1) or NPC2 (type C2) genes.

As used herein, a “neurological condition” is a disease that affects any part of the central or peripheral nervous system (e.g., brain, spine, and nerves).

As used herein, a “TMEM97 ligand” is any ligand that selectively and specifically binds to the TMEM97 receptor, specifically excluding ligands previously identified to bind the sigma-2 receptor (e.g., opipramol, MIN-101 (UNII-4P3110M3BF), CT-1812, siramesine, rimcazole, ibogaine, afobazole, BMY-14802 (alpha-(4-fluorophenyl)-4-(5-fluoro-2-pyrirnidinyl)-1-piperazine butanol), and panamesine).

As used herein, a “condition requiring neuroprotection” is any disease that results in the disruption of neuronal structure and/or function. Examples of conditions requiring neuroprotection are neurodegenerative diseases, stroke, asphyxiation, ischemia, intracranial aneurysm, myocardial infarction, spinal cord injury and head injury (e.g., concussion or traumatic brain injury).

### BRIEF DESCRIPTION OF THE DRAWINGS

FIG. 1 is a schematic diagram illustrating how the sigma-2 receptor was identified. The sigma-2 receptors were isolated from calves' livers and purified on an affinity resin of agarose beads covalently coupled to a sigma-2 ligand JVW-1625. The components that bound the resin were run on an SDS-PAGE gel and prepared for liquid chromatography and mass spectrometry analysis.

FIG. 2 is a graph showing ³H di-o-tolylguanidine (DTG) binding by HEK cells overexpressing 8 candidate sigma-2 receptor proteins and an empty vector control. Only TMEM97 showed significant ³H DTG binding.

FIG. 3 is a graph showing PC-12 cells treated with Tmem97-targeted or control siRNA to measure Tmem97 mRNA levels (left) and sigma-2 expression by ³H DTG binding (right). qPCR data are shown as mean+/−SD. Radioligand binding data are shown as mean+/−SEM and are of two representative experiments performed in triplicate.

FIG. 4 is graph showing a saturation binding curve of Sf9 insect cells expressing TMEM97 for ³H DTG, with a K_{d} of 11.3 nM.

FIG. 5 is graph showing a set of competition binding assays of known sigma-2 ligands including DTG, haloperidol, PB-28, (+)-pentazocine, (+)-SKF-10,047, and SAS-1121. In each case, the affinity of the ligand for TMEM97 was essentially identical to previously reported affinities.

FIG. 6 is a graph showing the results of a binding assay with two known TMEM97 ligands, Elacridar and Ro 48-8071 binding to the TMEM97 overexpressed in Sf9 insect cell membranes and MCF-7 cell membranes. In each case, the affinity of the ligand for TMEM97 was essentially identical to previously reported affinities.

FIG. 7 is a table showing the binding affinities of TMEM97 for various known sigma-2 and TMEM97 ligands.

FIG. 8 is a graph showing net ³H DTG binding by a number of sigma-2 receptor mutants. D29N and D56N mutations significantly impaired ³H DTG binding.

### DETAILED DESCRIPTION

This invention discloses new methods of treating cancer, sterol homeostasis diseases and neurological diseases in a subject (e.g. a patient) by administering pharmaceutical compositions in an amount and for a duration sufficient to treat the disease.

Since the 1970s, classical pharmacology and radioactive ligand binding assays have enabled the characterization of a plethora of cellular receptors for hormones, peptides, neurotransmitters, and other biologically active molecules. Among the receptors thus identified are the sigma receptors, which were first reported in 1976 and later classified into signa-1 and sigma-2 subtypes, based upon their differing affinities for (+) benzomorphans. Advances in molecular biology and receptor biochemistry led to the molecular clone of most pharmacologically-defined receptors, thereby enabling direct mapping of specific gene products to these sites and transforming our understanding of molecular pharmacology. Essentially all classically defined receptors were cloned by the mid-1990s, including the sigma-1 receptor, which was cloned in 1996. Subsequently, sigma-1-knockout mouse studies demonstrated that the pharmacologically similar sigma-2 “subtype” derives from a different, unknown gene. The identity of the gene encoding sigma-2 has continued to elude discovery despite almost thirty years of effort, making sigma-2 one of the last classical receptors remaining to be cloned.

The invention related to the identification of the sigma-2 receptor as transmembrane protein 97 (TMEM97). TMEM97, an ER-resident transmembrane protein has been recently implicated in sterol homeostasis disorders, such as Niemann-Pick disease. The cloning of the sigma-2 receptor resolves a longstanding pharmacological mystery and unites an emerging drug target with a number of established therapeutic molecules. Furthermore, because TMEM97 appears to be involved in sterol homeostasis, there is now a trove of ligands that had originally been identified as sigma-2 receptor binders that may be used to study and treat pathologies associated with aberrant cholesterol trafficking. Conversely, molecules that have been shown to target TMEM97 can now be applied to treat neurological diseases previously linked with the sigma-2 receptor. Additionally, all compounds can be used to treat cancer. New methods of treatment for cancer, sterol homeostasis diseases, and neurological diseases are hereby disclosed in this invention.

### Sigma-2 Receptor

In some embodiments of the invention, a disease is treated by targeting the sigma-2 receptor. The sigma-2 receptor is an 18-21 kDa membrane receptor located in lipid rafts that plays a role in hormonal, calcium, and neuronal signaling. The receptor can bind hormones and sterols (e.g. testosterone, progesterone, and cholesterol) and mediate signaling cascades via a calcium secondary messenger. High densities of the receptor can be found in the several areas of the CNS (e.g. cerebellum, motor cortex, hippocampus, substantia nigra, nucleus accumbens, central grey matter, olfactory bulb, subventricular zone, and oculomotor nucleus), liver, and kidney and the receptor is responsible for motor function and emotional response. The receptor is pharmacologically defined as a high-affinity binding site for di-o-tolylguanidine (DTG; Kᵢ=21.2 nM) and haloperidol (Kᵢ=48.7 nM), but with low affinity for (+)-benzomorphans. This contrasts with the sigma-1 receptor, which shows high affinity for all three compounds. The receptor has been shown to bind antipsychotic drugs (e.g. haloperidol, MIN-101), implicating it in a number of neuropsychiatric disorders associated with mood (affect) and emotional responses. Furthermore, the receptor is overexpressed in several cancer cell lines and proliferating tumors, rendering it a key cancer biomarker and potential therapeutic target. The sigma-2 receptor has never been previously cloned, and its gene has remained a mystery until now.

### TMEM97

In some embodiments of the invention, a disease is treated by targeting TMEM97. TMEM97 is a four pass ER-resident transmembrane protein that has been identified as a modulator of cholesterol levels. H. sapiens TMEM97, also known as MAC30, has the following cDNA and protein sequences:

| TMEM97 cDNA sequence (528) | |
|---|---|
| SEQ ID NO: 1 | |
| 1 | ATGGGGGCTC CGGCAACCAG GCGCTGCGTG GAGTGGCTGC TGGGCCTCTA CTTCCTCAGC |
| 61 | CACATCCCCA TCACCCTGTT CATGGACCTG CAGGCGGTGC TGCCGCGCGA GCTCTACCCA |
| 121 | GTCGAGTTTA GAAACCTGCT GAAGTGGTAT GCTAAGGAGT TCAAAGACCC ACTGCTACAG |
| 181 | GAGCCCCCAG CCTGGTTTAA GTCCTTTCTG TTTTGCGAGC TTGTGTTTCA GCTGCCTTTC |
| 241 | TTTCCCATTG CAACGTATGC CTTCCTCAAA GGAAGCTGCA AGTGGATTCG AACTCCTGCA |
| 301 | ATCATCTACT CTGTTCACAC CATGACAACC TTAATTCCGA TACTCTCCAC ATTTCTGTTT |
| 361 | GAGGATTTCT CCAAAGCCAG TGGTTTCAAG GGACAAAGAC CTGAGACTTT GCATGAACGG |
| 421 | TTAACCCTTG TGTCTGTCTA TGCCCCCTAC TTACTCATCC CATTCATACT TTTAATTTTC |
| 481 | ATGTTGCGGA GCCCCTACTA CAAGTATGAA GAGAAAAGAA AAAAAAAA |

TMEM97 protein sequence (176)
SEQ ID NO: 2
MGAPATRRCVEWLLGLYFLSHIPITLFMDLQAVLPRELYPVEFRNLLKWYAKEFKDPLLQEPPAWFKS
FLFCELVFQLPFFPIATYAFLKGSCKWIRTPAIIYSVHTMTTLIPILSTFLFEDFSKASGFKGQRPET
LHERLTLVSVYAPYLLIPFILLIFMLRSPYYKYEEKRKKK

TMEM97 has been shown to interact with NPC1, the protein associated with Niemann-Pick disease type C1. Reduction of TMEM97 (e.g., via siRNA knockdown) increases NPC1 protein levels in NPC cell models and in fibroblasts from NPC1 patients. This functions to counteract lysosomal lipid accumulation and restore normal sterol homeostasis, therefore making it an attractive target for NPC therapeutics.

### Sterol Homeostasis Diseases

Sterol homeostasis diseases are conditions that disrupt the normal equilibrium of natural steroid alcohols in the cell. These diseases may be caused, for example, by disruption of sterol transport or sterol biogenesis. Exemplary sterol homeostasis diseases are Niemann-Pick disease and Smith-Lemli-Opitz syndrome (SLOS).

### Niemann-Pick Disease

Niemann-Pick disease is a metabolic disorder in which sphingolipids accumulate in cell lysosomes.

Lysosomes are responsible for transportation of material in and out of cells, while mutations that disrupt this process cause the disease. Niemann-Pick disease is commonly divided into four subtypes, type A (NPA), B (NPB), C1 (NPC1), and C2 (NPC2). NPA and NPB are associated with mutations in the SMPD1 gene, a sphingomyelin phosphodisesterase, while mutations in the NPC1 and NPC2 genes are associated with NPC1 and NPC2, respectively. NPC1 and NPC2 function as a tag team of membrane proteins that mediate intracellular cholesterol trafficking in mammals. NPC2 binds cholesterol that has been released in the endosomal lumen and transfers it to the cholesterol-binding pocket of the N-terminal domain of NPC1. NPC1 then exports the cholesterol to the ER and plasma membranes. Thus, loss of or mutations in either of NPC1 or NPC2 perturbs this transportation process and disrupts normal cholesterol homeostasis.

Niemann-Pick disease is inherited and autosomally recessive. Thus, two defective copies of the gene are required for manifestation of the disease. Common symptoms include enlargement of the liver and spleen due to accumulation of sphingomyelin, low platelet count, and persistent lung infection. Furthermore, accumulation of sphingomyelin in the central nervous system (CNS) can result in seizures, ataxia, dysarathria, dysphagia, and a number of other cognitive and physical impairments. NPA is usually childhood lethal by 18 months, NPB presents itself in mid-childhood with survival into adulthood, while NPC1 and NPC2 presents later with some surviving into adulthood. Currently, no effective therapeutics exist for the disease, with most treatments focusing on symptomatic relief.

### Neurological Conditions

Neurological conditions are diseases that affect any part of the central nervous system (CNS) or peripheral nervous system (PNS). Exemplary neurological conditions are conditions requiring neuroprotection, stroke, anxiety, depression, Alzheimer's disease, Frontotemporal dementia, Lewy Body dementia, Pick's disease, Huntington's disease, pain, Parkinson's disease, multiple sclerosis, microglia inflammation, schizophrenia, addiction, and head injury (e.g., concussion or traumatic brain injury). Examples of neurological conditions include pain, neuropathic pain, and addiction (e.g., addiction to opioid, cocaine, methamphetamine, and alcohol).

### Cancer

Cancer is a group of diseases involving abnormal cell growth with the potential to invade or spread to other parts of the body. Exemplary cancers include leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer, acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia. In some preferred embodiments, the methods of this invention can be used to treat, for example, sqamous cell carcinoma, glioma, colorectal cancer, gastric cancer, epitherlial ovarian cancer, non-small-cell lung cancer, and breast cancer.

### Ligands

Targeting a receptor for disease treatment may be accomplished, for example, by providing a ligand, which binds to the targeted receptor. A ligand is an ion, small molecule, or protein that selectively or specifically binds to a certain receptor (e.g. protein receptor) and modulates its activity. Binding of a ligand may induce a conformational change in its target to produce a biological or physiological response. For example, ibogaine can bind the sigma-2 receptor to set off a signaling cascade that leads to dopamine release. Binding occurs by intermolecular forces (e.g. ionic bonds, hydrogen bonds, VDW forces) and is usually reversible in nature. Ligand binding to a receptor is characterized by a binding affinity dissociation constant K_{d} or inhibition constant Kᵢ. Higher affinity ligands have a lower dissociation constant, and therefore a higher degree of occupancy than low affinity ligands. A ligand that triggers a physiological response is an agonist for the receptor. A ligand that inhibits a physiological response is an antagonist, while a ligand that produces an intermediate response is a partial agonist.

In some embodiments, a ligand can be a protein, such as a hormone or antibody. An antibody is an immunoglobulin protein that specifically binds a target antigen. Antibodies are composed of two copies each of a heavy and light chain (VH and VL). At the N-termini of each chain are the complementarity determining regions (CDRs) that impart specific and selective binding properties to the antibody. An antibody can function as a ligand wherein it binds to a receptor to trigger a physiological response.

### Sigma-2 Receptor Ligands

Sigma-2 receptor ligands include for example, small molecules that have been previously described in the art. A sigma-2 receptor agonist has been described in the art as a ligand that has at least 90% of the cytotoxic activity of siramesine in a tumor cell apoptosis assay with EMT-6 and human melanoma cell line MDA-MB-435. An antagonist has less than 10% of the cytotoxic activity of siramesine, while a partial agonist has between 10% and 90% of the cytotoxic activity of siramesine.

Exemplary sigma-2 receptor ligands are opipramol, MIN-101 (2-[[1-[2-(4-fluorophenyl)-2-oxoethyl]piperidin-4-yl]methyl]-3H-isoindol-1-one), CT-1812, siramesine, rimcazole, ibogaine, afobazole, BMY-14802 (1-(4-Fluorophenyl)-4-[4-(5-fluoro-2-pyrimidinyl)-1-piperazinyl]-1-butanol), and panamesine. The foregoing ligands are described, e.g., in German Federal Republic Patent No. 1,132,556, U.S. Pat. Nos. 9,458,130, 7,166,617, 8,765,816, PCT Publication No. WO 15/116923, U.S. Pat. Nos. 5,665,725, 4,379,160, 4,499,096, Russian Patent No. 2,061,686, Russian Patent No. 2,485,954, U.S. Pat. Nos. 4,605,655, and 5,232,931, the disclosure of each of which is incorporated herein by reference it pertains to sigma-2 receptor ligands.

Additional exemplary sigma-2 receptor ligands are ¹¹C-PB-28, ¹²⁵I RHM-4, ¹²⁵I-IAC44, ¹²⁵I-IAF(1-N-(2′,6′-dimethyl-morpholino)-3-(4-azido-3-[(125)I]iodo-phenyl)propane, ¹⁸F ISO-1, 2-(4-(3-(4-fluorophenyl)indol-1-yl)butyl)-6,7-dimethoxy-1,2,3,4-tetrahydroisoquinoline), ³H DTG, ³H-azido-DTG, ³H-PB28, ³H-RHM-1, ^{99m}Tc BAT-EN6, ^{99m}Tc-4-(4-cyclohexylpiperazine-1-yl)-butan-1-one-1-cyclopentadienyltricarbonyl technetium, ABN-1, AG-205, ANSTO-19, benzoxazolone, BIMU-1, CB-182, CB-184, CB-64D, CB-64L, cocaine, ditolylguanidine (DTG), F281, indole ((1-[3-[4-(substituted-phenyl) piperazin-1-yl]-propyl]-1H-indole, K05-138, K05-138, N-Benzyl-7-azabicyclo[2.2.1]heptane, PB183, PB28, RHM-1, RHM-138, RHM-2, RHM-4, SM-21, SN79, SV119, SW107, SW116, SW120, SW43, TC4ANSTO-19, WC-21, WC-26, WC-59, yun179, yun194, yun201, yun202, yun203, yun204, yun209, yun210, yun212, yun234, yun236, yun242, yun243 (RMH-1), yun245, yun250, yun251, yun253, yun254, and yun552. The foregoing sigma-2 receptor ligands are described e.g., in Curr Med Chem. 2015; 22:989-1003, J Med Chem. 2013; 56:7137-60, and Med Res Rev. 2014; 34:532-66, the disclosure of each of which is incorporated herein by reference in its entirety.

Additional exemplary sigma-2 receptor ligands include SAS-0132, DKR-1051, DKR-1005, JVW-1009, and any additional compounds described in J Neurochem. 2017 February; 140(4):561-575, which is incorporated herein by reference in its entirety for all purposes.

Additional exemplary sigma-2 receptor ligands include compounds 12, 16, 20, 39, 40, 19, 38, 27, 41, 42, 43, 44, 32, 33, 34, 35, 36, 37, 28, 29, 30, 31 (SAS-1121), and any additional compounds described in ChemMedChem. 2016 Mar. 17; 11(6):556-61, which is incorporated herein by reference in its entirety for all purposes.

Additional exemplary sigma-2 receptor ligands are compounds 7, 10, 11, 13, 14, 15, 18, 19, 20, 21, 22, 24, 25, 27, 28, 29, 30, 32, 33, 34, 36, and 46, as described in Curr Med Chem. 2015; 22(8):989-1003, compounds 9, 10, 11, 12, 13, 15, 19, 20, 21, 22, 23, 24, 25, 26, 27, 28, 29, 30, 31, 32, 33, 34, 35, 36, 38, 39, 40, 42, 44, 47, 51, 56, and 57, as described in J Med Chem. 2013 Sep. 26; 56(18):7137-60, and compounds 8, 9, 10, 11, 13, 14, 15, 16, 17, 20, 21, 22, 26, 29, 30, 31, 32, 33, 34, 36, 37, 38, 39, 40, 41, 42, 43, 47, 50, 51, 52, 53, 54, 55, 56, 57, 58, 59, 60, 67, 69, 70, 71, 72, 73, 74, 75, 76, 77, 78, 79, 80, 81, 82, 83, 84, 85, 86, 87, 88, 89, 90, 92, 93, 94, 96, 97, 98, 99, 101, 102, 103, 104, 113, 115, and 116, as described in Med Res Rev. 2014; 34:532-66.

Additional sigma-2 receptor ligands are disclosed in US Patent Application Publication No. 2006/0004036, US Patent Application Publication No. 2012/0190710, US Patent Application Publication No. 2013/0274290, PCT Publication No. WO 01/85153, PCT Publication No. WO 01/80905, PCT Publication No. WO 97/34892, PCT Publication No. WO 97/30038, PCT Publication No. WO 96/05185, EP Patent Publication No. 0881220, U.S. Pat. Nos. 6,015,543, 5,993,777, 5,919,934, 5,969,138, 5,911,970, and PCT Publication No. WO 01/85153, the disclosure of each of which is incorporated herein by reference it pertains to sigma-2 receptor ligands.

In some embodiments, the sigma-2 receptor ligand is a compound having the formula:

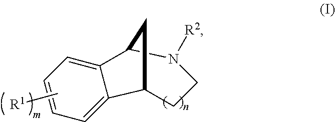

wherein:
R¹ is hydrogen, halogen (e.g., —Cl), —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —N₃, —CF₃, —CCl₃, —CBr₃, —CCI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n1 and n2 are independently 1 or 2;
m is 1, 2, 3 or 4;
n is 1 or 2; and
R³, R^{3A}, R⁴, R^{4A} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
In embodiments, R² is hydrogen, —CF₃, —CN, —C(O)R⁴, —OR⁴, —NR⁴R^{4A}, —C(O)OR⁴, —C(O)NR⁴R^{4A}, —NO₂, —S(O)ₙ₂R⁴, —S(O)ₙ₂OR⁴, —S(O)ₙ₂NR⁴R^{4A}, —NHNR⁴R^{4A}, —NHC(O)NHNR⁴R^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, the ligand is a compound having the formula:

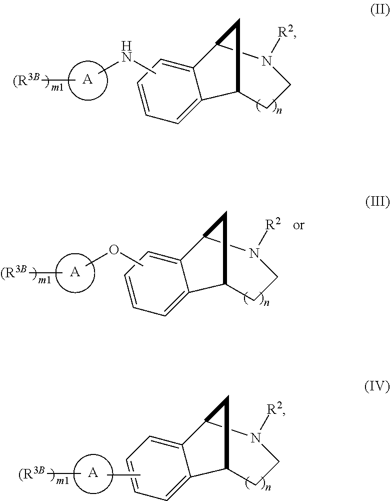

wherein
R^{3B} is —CF₃, —CN, —OH, —NH₂, —CONH₂, —S(O)₃H, —S(O)₂NH₂, —NHC(O) NH₂, —NHC(O)H, —OCHF₂, oxo, halogen, —COOH, —NO₂, —SH, —S(O)₄H, —NHNH₂, —ONH₂, —NHC(O)NHNH₂, —NHS(O)₂H, —NHC(O)—OH, —NHOH, —OCF₃, unsubstituted alkyl, unsubstituted heteroalkyl, unsubstituted cycloalkyl, unsubstituted heterocycloalkyl, unsubstituted aryl or unsubstituted heteroaryl;
ring A is aryl, heteroaryl, cycloalkyl or heterocycloalkyl; and
m1 is 0, 1, 2, 3, or 4.

In some embodiments, the ligand is a compound having the formula:

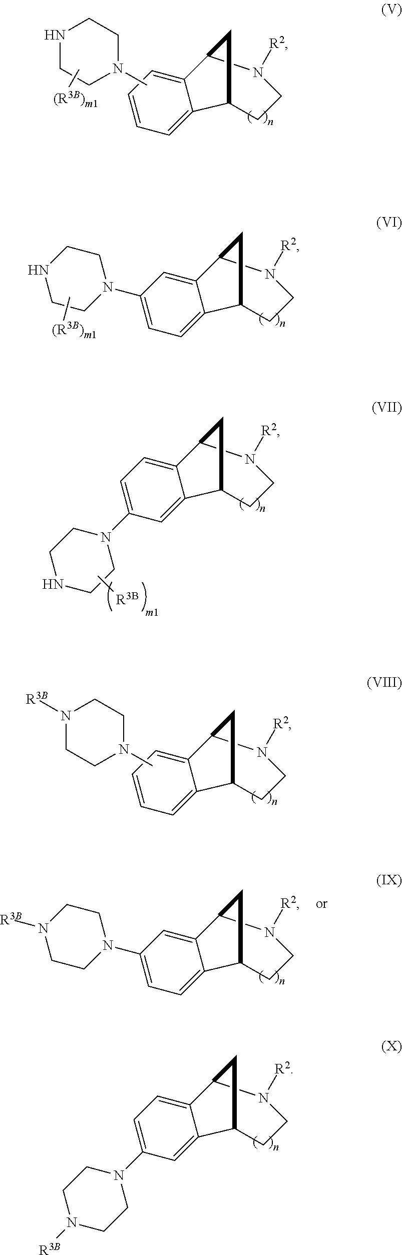

In some embodiments, the ligand is a compound having the formula:

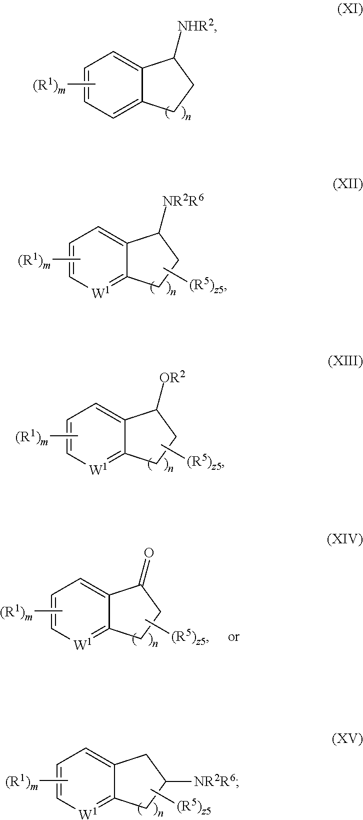

wherein R¹ is hydrogen, halogen (e.g., —F, —Cl, —Br, —I), —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, substituted or unsubstituted alkyl, substituted or unsubstituted heteroalkyl, substituted or unsubstituted cycloalkyl, substituted or unsubstituted heterocycloalkyl (e.g., piperazinyl, piperidinyl, morpholinyl), substituted or unsubstituted aryl (e.g., phenyl), or substituted or unsubstituted heteroaryl (e.g., pyridyl); R² is hydrogen, halogen, —N₃, —CF₃, —CCl₃, —CBr₃, —CI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, substituted or unsubstituted alkyl (e.g., —CH₃, —CH₂CH₃, —CH₂CH₂CH₃, —CH₂CH₂CH₂OH, —CH₂Ph), substituted or unsubstituted heteroalkyl (e.g., —C(O)OCH₂Ph, —C(O)NHCH₂Ph, —CH₂CH₂C(O)OCH₂CH₃, —CH₂CH₂C(O)OCH₃, —CH₂CH₂OCH₂CH₃, —CH₂CH₂OCH₃), substituted or unsubstituted cycloalkyl, substituted or unsubstituted heterocycloalkyl (e.g., tetrahydropyranyl, piperidinyl, methyl substituted piperidinyl), substituted or unsubstituted aryl, or substituted or unsubstituted heteroaryl; the symbols n1 and n2 are independently 1 or 2; the symbol m is 1, 2, 3 or 4; n is 1, 2, 3 or 4; R³, R^{3A}, R⁴, R^{4A} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is halogen, —N₃, —CF₃, —CCl₃, —CBr₃, —CI₃, —CN, —C(O)R^{5C}, —OR^{5D} (e.g., —OH), —NR^{5A}R^{5B}, —C(O)OR^{5D}, —C(O)NR^{5A}R^{5B}, —NO₂, —SR^{5D}, —S(O)ₙ₅R^{5C}, —S(O)ₙ₅OR^{5D}, —S(O)ₙ₅NR^{5A}R^{5B}, —NHNR^{5A}R^{5B}, —ONR^{5A}R^{5B}, —NHC(O)NHNR^{5A}R^{5B}, substituted or unsubstituted alkyl (e.g., —CH₂Ph), substituted or unsubstituted heteroalkyl, substituted or unsubstituted cycloalkyl, substituted or unsubstituted heterocycloalkyl, substituted or unsubstituted aryl, or substituted or unsubstituted heteroaryl; the symbol n5 is independently 1 or 2; the symbol z5 is independently an integer from 0 to 6; R⁶ is halogen, —N₃, —CF₃, —CCl₃, —CBr₃, —CI₃, —CN, —C(O)R^{6C}, —OR^{6D}, —NR^{6A}R^{6B}, —C(O)OR^{6D}, —C(O)NR^{6A}R^{6B}, —NO₂, —SR^{6D}, —S(O)ₙ₆R^{6C}, —S(O)ₙ₆OR^{6D}, —S(O)ₙ₆NR^{6A}R^{6B}, —NHNR^{6A}R^{6B}, —ONR^{6A}R^{6B}, —NHC(O)NHNR^{6A}R^{6B}, substituted or unsubstituted alkyl (e.g., —CH₃, —CH₂CH₃, —CH₂CH₂CH₃, —CH₂CH₂CH₂OH, —CH₂Ph), substituted or unsubstituted heteroalkyl (e.g., —C(O)OCH₂Ph, —CH₂CH₂C(O)OCH₂CH₃, —CH₂CH₂C(O)OCH₃, —CH₂CH₂OCH₂CH₃, —CH₂CH₂OCH₃), substituted or unsubstituted cycloalkyl, substituted or unsubstituted heterocycloalkyl (e.g., tetrahydropyranyl, piperidinyl, methyl substituted piperidinyl), substituted or unsubstituted aryl, or substituted or unsubstituted heteroaryl; the symbol n6 is independently 1 or 2; W¹ is CH, C(R¹), or N; and R^{5A}, R^{5B}, R^{5C}, R^{5D}, R^{6A}, R^{6B}, R^{6C} and R^{6D} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
In embodiments, R² is hydrogen, —CF₃, —CN, —C(O)R⁴, —OR⁴, —NR⁴R^{4A}, —C(O)OR⁴, —C(O)NR⁴R^{4A}, —NO₂, —S(O)ₙ₂R⁴, —S(O)ₙ₂OR⁴, —S(O)ₙ₂NR⁴R^{4A}, —NHNR⁴R^{4A}, —NHC(O)NHNR⁴R^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. If used in the context of a larger list of chemical groups wherein unsaturated alkyl groups are specifically defined then the term “alkyl” is used to describe a saturated group. An unsaturated alkyl group may be further refined as alkenyl which is an unsaturated alkyl group with one or more carbon-carbon double bonds and no carbon-carbon triple bonds. Similarly, an unsaturated alkyl group may be further refined as alkynyl which is an unsaturated alkyl group with one or more carbon-carbon triple bonds. An alkynyl group may contain one or more carbon-carbon double bonds so long as it contains at least one carbon-carbon triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). Similarly, an aralkyl group is a substituted alkyl group which has been substituted with one or more aryl groups as this term is described herein. These aralkyl group may be substituted as described below in agreement with the common chemical bonding valency. Some non-limiting examples of unsubstituted aralkyl groups include benzyl, phenylethyl, and diphenylethyl. Furthermore, an aralkenyl group is a subset wherein the substituted alkyl group is an alkenyl group as that term has been defined above.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., selected from the group consisting of O, N, P, Si, and S, and wherein the nitrogen and sulfur atoms may optionally be oxidized, and the nitrogen heteroatom may optionally be quaternized). The heteroatom(s) (e.g., O, N, P, S, B, As, and Si) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. These groups include the possibility that one or more of these groups may have one or more saturated alkyl substitutions on the ring system provided that the point of connection is the ring system.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be a —O-bonded to a ring heteroatom nitrogen.

A “fused ring aryl-heterocycloalkyl” is an aryl fused to a heterocycloalkyl. A “fused ring heteroaryl-heterocycloalkyl” is a heteroaryl fused to a heterocycloalkyl. A “fused ring heterocycloalkyl-cycloalkyl” is a heterocycloalkyl fused to a cycloalkyl. A “fused ring heterocycloalkyl-heterocycloalkyl” is a heterocycloalkyl fused to another heterocycloalkyl. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be unsubstituted or substituted with one or more of the substituents described herein. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be named according to the size of each of the fused rings. Thus, for example, 6,5 aryl-heterocycloalkyl fused ring describes a 6 membered aryl moiety fused to a 5 membered heterocycloalkyl. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

The term “thio,” as used herein, means a sulfur that is single or double bonded to carbon, or single bonded to another sulfur.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, a substitutent group as that term is defined below or —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like). In some embodiments, the substitution may include the removal of one or more hydrogen atom and replacing it with one of the following groups: —OH, —F, —Cl, —Br, —I, —NH₂, —NO₂, —CO₂H, —CO₂CH₃, —CO₂CH₂CH₃, —CN, —SH, —OCH₃, —OCF₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —NHC(O)NH₂, —S(O)₂OH, —S(O)₂CH₃, or —S(O)₂NH₂.

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present. In some embodiments, the substitution may include the removal of one or more hydrogen atom and replacing it with one of the following groups: —OH, —F, —Cl, —Br, —I, —NH₂, —NO₂, —CO₂H, —CO₂CH₃, —CO₂CH₂CH₃, —CN, —SH, —OCH₃, —OCF₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —NHC(O)NH₂, —S(O)₂OH, —S(O)₂CH₃, or —S(O)₂NH₂.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroalyl.

As used herein, the terms “heteroatom” or “ring heteroatom” are meant to include, oxygen (O), nitrogen (N), sulfur (S), phosphorus (P), Boron (B), Arsenic (As), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

Unless otherwise defined herein, the chemical groups used herein may contain between 1 to 20 carbon atoms or ring members. In some preferred embodiments, the chemical group contains 1 to 12 carbon atoms or ring members. In more preferred embodiments, the chemical group contains 1 to 8 carbon atoms or ring members.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl, alkenyl, alkynyl, aryl, aralkyl, or aralkenyl may be a substituted or unsubstituted C₁-C₂₀ alkyl, alkenyl, alkynyl, aryl, aralkyl, or aralkenyl each substituted or unsubstituted heteroalkyl, heteroaryl, heteroaralkyl, or heteroaralkenyl is a substituted or unsubstituted 2 to 20 membered heteroalkyl, heteroaryl, heteroaralkyl, or heteroaralkenyl, each substituted or unsubstituted cycloalkyl or cycloalkenyl is a substituted or unsubstituted C₃-C₈ cycloalkyl or cycloalkenyl, and/or each substituted or unsubstituted heterocycloalkyl is a substituted or unsubstituted 3 to 8 membered heterocycloalkyl. In some embodiments of the compounds herein, each substituted or unsubstituted alkylene, alkenylene, alkynylene, arylene, aralkylene, or aralkenylene is a substituted or unsubstituted C₁-C₂₀ alkylene, alkenylene, alkynylene, arylene, aralkylene, or aralkenylene, each substituted or unsubstituted heteroalkylene, heteroarylene, heteroaralkylene, or heteroaralkenylene is a substituted or unsubstituted 2 to 20 membered heteroalkylene, heteroarylene, heteroaralkylene, or heteroaralkenylene, each substituted or unsubstituted cycloalkylene or cycloalkenylene is a substituted or unsubstituted C₃-C₈ cycloalkylene or cycloalkenylene, and/or each substituted or unsubstituted heterocycloalkylene is a substituted or unsubstituted 3 to 8 membered heterocycloalkylene.

In some embodiments, each substituted or unsubstituted alkyl, alkenyl, alkynyl, aryl, aralkyl, or aralkenyl is a substituted or unsubstituted C₁-C₈ alkyl, alkenyl, alkynyl, aryl, aralkyl, or aralkenyl, each substituted or unsubstituted heteroalkyl, heteroaryl, heteroaralkyl, or heteroaralkenyl is a substituted or unsubstituted 2 to 8 membered heteroalkyl, heteroaryl, heteroaralkyl, or heteroaralkenyl each substituted or unsubstituted cycloalkyl or cycloalkenyl is a substituted or unsubstituted C₃-C₇ cycloalkyl or cycloalkenyl, and/or each substituted or unsubstituted heterocycloalkyl is a substituted or unsubstituted 3 to 7 membered heterocycloalkyl. In some embodiments, each substituted or unsubstituted alkylene, alkenylene, alkynylene, arylene, aralkylene, or aralkenylene is a substituted or unsubstituted C₁-C₈ alkylene, alkenylene, alkynylene, arylene, aralkylene, or aralkenylene, each substituted or unsubstituted heteroalkylene, heteroarylene, heteroaralkylene, or heteroaralkenylene is a substituted or unsubstituted 2 to 8 membered heteroalkylene, heteroarylene, heteroaralkylene, or heteroaralkenylene, each substituted or unsubstituted cycloalkylene or cycloalkenylene is a substituted or unsubstituted C₃-C₇ cycloalkylene or cycloalkenylene, and/or each substituted or unsubstituted heterocycloalkylene is a substituted or unsubstituted 3 to 7 membered heterocycloalkylene.

The small molecule ligands of the present invention are shown, for example, above, in the summary of the invention section, and in the claims below. They may be made using the synthetic methods outlined in the Examples section or as described in references cited herein. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch of continuous, using the principles and techniques of process chemistry as applied by a person skilled in the art. Such principles and techniques are taught, for example, in Anderson, Practical Process Research & Development A Guide for Organic Chemists (2012), which is incorporated by reference herein.

All of the ligands of the present invention may be useful for the prevention and treatment of one or more diseases or disorders discussed herein or otherwise. In some embodiments, one or more of the ligands characterized or exemplified herein as an intermediate, a metabolite, and/or prodrug, may nevertheless also be useful for the prevention and treatment of one or more diseases or disorders. As such unless explicitly stated to the contrary, all of the ligands of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the ligands of the present invention have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, ligands known in the prior art, whether for use in the indications stated herein or otherwise.

Ligands of the present invention may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Ligands may occur as racemates and racemic mixtures, single enantiomers, diastereomeric mixtures and individual diastereomers. In some embodiments, a single diastereomer is obtained. The chiral centers of the ligands of the present invention can have the S or the R configuration.

Chemical formulas used to represent ligands of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given ligand, and regardless of which one is most prevalent, all tautomers of a given chemical formula are intended.

In addition, atoms making up the ligands of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The ligands of the present invention may also exist in prodrug form. Since prodrugs are known to enhance numerous desirable qualities of pharmaceuticals (e.g., solubility, bioavailability, manufacturing, etc.), the ligands employed in some methods of the invention may, if desired, be delivered in prodrug form. Thus, the invention contemplates prodrugs of ligands of the present invention as well as methods of delivering prodrugs. Prodrugs of the ligands employed in the invention may be prepared by modifying functional groups present in the compound in such a way that the modifications are cleaved, either in routine manipulation or in vivo, to the parent ligand. Accordingly, prodrugs include, for example, ligands described herein in which a hydroxy, amino, or carboxy group is bonded to any group that, when the prodrug is administered to a subject, cleaves to form a hydroxy, amino, or carboxylic acid, respectively.

Additional examples of pharmaceutically acceptable salts and their methods of preparation and use are presented in Handbook of Pharmaceutical Salts: Properties, and Use (2002), which is incorporated herein by reference.

It will appreciated that many of the ligands can form complexes with solvents in which they are reacted or from which they are precipitated or crystallized. These complexes are known as “solvates.” Where the solvent is water, the complex is known as a “hydrate.” It will also be appreciated that many ligands can exist in more than one solid form, including crystalline and amorphous forms. All solid forms of the ligands provided herein, including any solvates thereof are within the scope of the present invention.

In some embodiments, the ligand is JVW-1601 or JVW-1625:

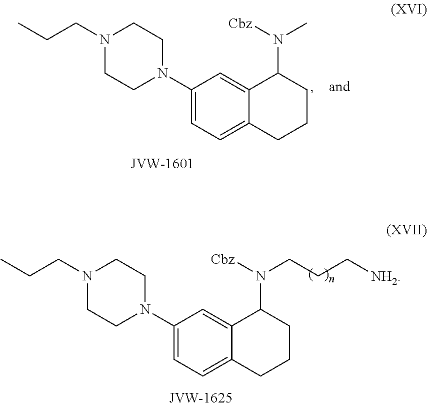

### TMEM97 Ligands

TMEM97 ligands include small molecules that have been previously described in the art. Exemplary ligands are elacridar and Ro 48-8071. Exemplary TMEM97 ligands are described, e.g., in U.S. Pat. Nos. 5,604,237 and 5,495,048, the disclosures of which are incorporated herein by reference as they pertain to TMEM97 ligands.

### RNA Therapies

Targeting a receptor for disease treatment may be accomplished, for example, by providing an RNA molecule, which modulates the function or expression of the receptor. The use of RNA as a therapeutic has been well established in the art. RNA therapeutics function to modulate protein expression (e.g. lower protein expression, abolish protein expression) in order to abrogate the effect of a deleterious protein. RNA therapeutics include, for example, microRNA (miRNA), small interfering RNA (siRNA), and antisense RNA. miRNA is a small non-coding RNA, usually containing 22 nucleotides that functions in RNA silencing and post-transcriptional regulation of gene expression. miRNAs function by base-pairing with complementary sequences within mRNA molecules, and silencing the mRNA by cleavage of the mRNA into two pieces, destabilization of the mRNA through shortening the poly(A) tail, or reducing the translation efficiency of the mRNA by ribosomes. siRNA is a 20-25 bp double stranded RNA that functions via a similar pathway as miRNA. siRNA interferes with the expression of specific genes with commentary nucleotide sequences by degrading mRNA after transcription, thereby abrogating translation. Antisense RNA is single stranded RNA that hybridizes to complementary mRNA by base pairing to it and physically obstructing the translation machinery.

### Methods of Treatment

### Formulations and Carriers

This invention describes methods of treatment for sterol homeostasis and neurological diseases by administering a pharmaceutical composition. The pharmaceutical composition can be formulated with a pharmaceutically acceptable carrier or excipient. A pharmaceutically acceptable carrier or excipient refers to a carrier (e.g., carrier, media, diluent, solvent, vehicle, etc.) which does not significantly interfere with the biological activity or effectiveness of the active ingredient(s) of a pharmaceutical composition and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types).

A pharmaceutical composition is typically formulated to be compatible with its intended route of administration. For oral administration, agents can be formulated by combining the active compounds with pharmaceutically acceptable carriers well known in the art. Such carriers enable the compounds of the invention to be formulated as a powder, tablet, pill, capsule, lozenge, liquid, gel, syrup, slurry, suspension, and the like. It is recognized that some pharmaceutical compositions, if administered orally, must be protected from digestion. This is typically accomplished either by complexing the protein with a composition to render it resistant to acidic and enzymatic hydrolysis or by packaging the protein in an appropriately resistant carrier such as a liposome. Suitable excipients for oral dosage forms include, for example, fillers such as sugars, including lactose, sucrose, mannitol, or sorbitol; cellulose preparations such as, for example, starch, gelatin, gum tragacanth, methyl cellulose, hydroxypropylmethyl cellulose, sodium carboxymethylcellulose, and/or polyvinylpyrrolidone (PVP). Disintegrating agents may be added, for example, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

For administration by inhalation, pharmaceutical compositions may be formulated in the form of an aerosol spray from a pressured container or dispenser, which contains a suitable propellant, e.g., a gas such as carbon dioxide, a fluorocarbon, or a nebulizer. Liquid or dry aerosol (e.g., dry powders, large porous particles, etc.) can also be used. For topical application, a pharmaceutical composition may be formulated in a suitable ointment, lotion, gel, or cream containing the active components suspended or dissolved in one or more pharmaceutically acceptable carriers suitable for use in such compositions

### Dosage and Administration

The pharmaceutical compositions used in this invention can be administered to a subject (e.g. patient) in a variety of ways. The compositions must be suitable for the subject receiving the treatment and the mode of administration. Furthermore, the severity of the disease to be treated affects the dosages and routes. The pharmaceutical compositions used in this invention can be administered orally, sublingually, parenterally, intravenously, subcutaneously, intramedullary, intranasally, as a suppository, using a flash formulation, topically, intradermally, subcutaneously, via pulmonary delivery, via intra-arterial injection, or via a mucosal route.

In some embodiments, the effective dose range for the therapeutic compound can be extrapolated from effective doses determined in animal studies for a variety of different animals. In general a human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some particular embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

In general, the dosage of a pharmaceutical composition be in the range of from about 1 ng to about 1 kg (e.g. 1 ng-10 ng, e.g, 2 ng, 3 ng, 4 ng, 5 ng, 6 ng, 7 ng, 8 ng, 9 ng, 10 ng, e.g., 10 ng-100 ng, e.g., 20 ng, 30 ng, 40 ng, 50 ng, 60 ng, 70 ng, 80 ng, 90 ng, 100 ng, e.g., 100 ng-1 μg, e.g., 200 ng, 300 ng, 400 ng, 500 ng, 600 ng, 700 ng, 800 ng, 900 ng, 1 μg, e.g. 1-10 μg, e.g. 1 μg, 2 μg, 3 μg, 4 μg, 5 μg, 6 μg, 7 μg, 8 μg, 9 μg, 10 μg, e.g., 10 μg-100 μg, e.g., 20 μg, 30 μg, 40 μg, 50 μg, 60 μg, 70 μg, 80 μg, 90 μg, 100 μg, e.g., 100 μg-1 mg, e.g., 200 μg, 300 μg, 400 μg, 500 μg, 600 μg, 700 μg, 800 μg, 900 μg, 1 mg, e.g., 1 mg-10 mg, e.g., 2 mg, 3 mg, 4 mg, 5 mg, 6 mg, 7 mg, 8 mg, 9 mg, 10 mg, e.g. 10 mg-100 mg, e.g., 20 mg, 30 mg, 40 mg, 50 mg, 60 mg, 70 mg, 80 mg, 90 mg, 100 mg, e.g., 100 mg-1 g, e.g., 200 mg, 300 mg, 400 mg, 500 mg, 600 mg, 700 mg, 800 mg, 900 mg, 1 g, e.g., 1 g-10 g, e.g. 2 g, 3 g, 4 g, 5 g, 6 g, 7 g, 8 g, 9 g, 10 g, e.g., 10 g-100 g, e.g., 20 g, 30 g, 40 g, 50 g, 60 g, 70 g, 80 g, 90 g, 100 g, e.g., 100 g-1 kg, e.g., 200 g, 300 g, 400 g, 500 g, 600 g, 700 g, 800 g, 900 g, 1 kg).

The dosage regimen may be determined by the clinical indication being addressed, as well as by various patient variables (e.g. weight, age, sex) and clinical presentation (e.g. extent or severity of disease). Furthermore, it is understood that all dosages may be continuously given or divided into dosages given per a given time frame. The composition can be administered, for example, every hour, day, week, month, or year.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1. Identification of the Sigma-2 Receptor

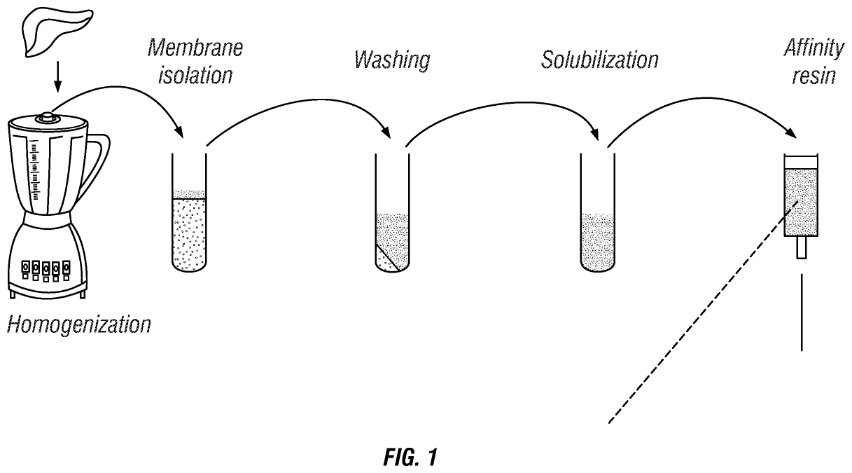

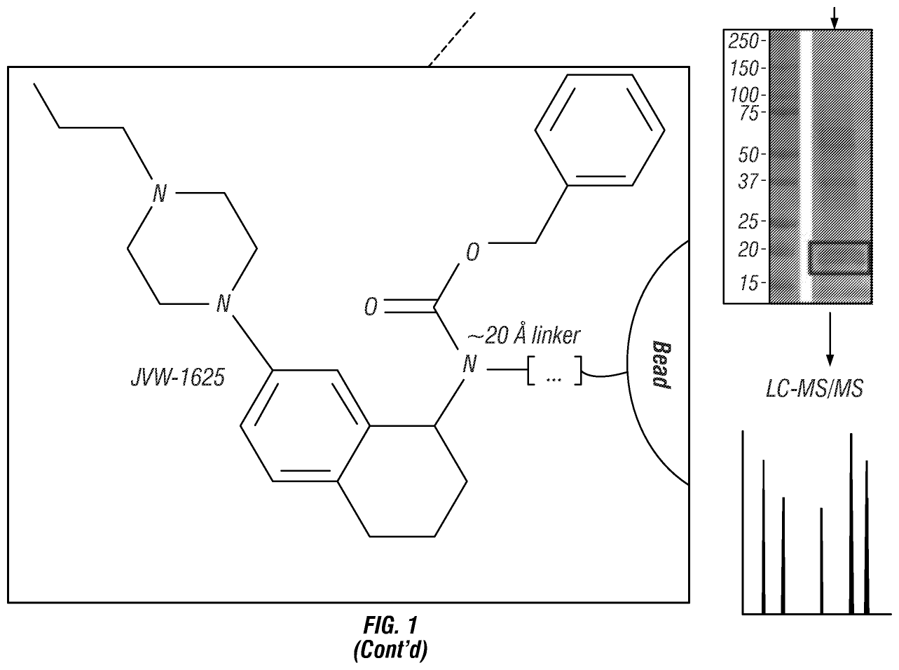

The sigma-2 receptor was identified using a biochemical approach. First, JVW-1625, a high affinity sigma-2 receptor ligand derived from JVW-1601, was synthesized. JVW-1625 was covalently coupled to agarose beads to prepare an affinity chromatography resin (FIG. 1). The sigma-2 receptor was isolated from homogenized calves' livers. Following homogenization, the membranes were isolated, washed, and solubilized before passing over the affinity resin. The contents that bound the affinity resin were analyzed by SDS-PAGE and LC-MS/MS.

### Example 2. Characterizing the Sigma-2 Receptor

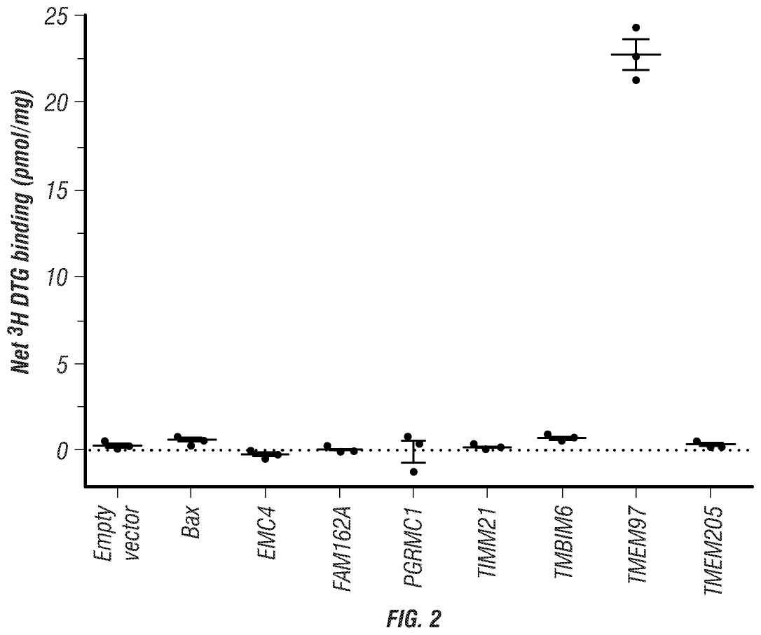

Seven candidate proteins were identified that bound the affinity resin. All candidates and PGRMC1 were overexpressed in HEK293 cells and assayed for their ability to bind ³H DTG (10 nM concentration), a core pharmacological feature of the sigma-2 receptor. Of the 8 candidates, only TMEM97 showed significant binding of ³H DTG (FIG. 2). PGRMC1 overexpression did not lead to increased ³H DTG binding.

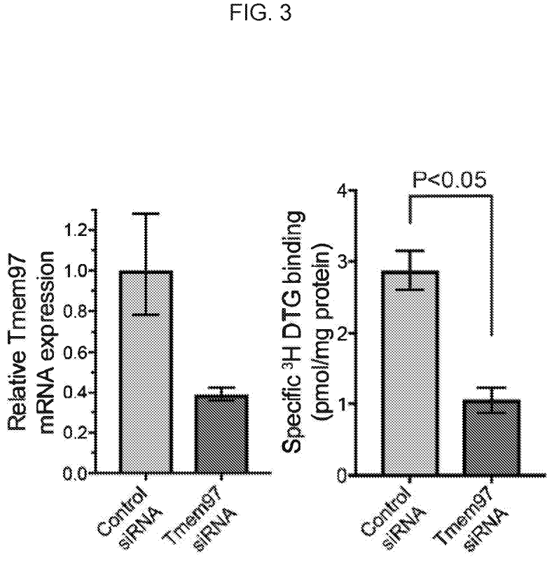

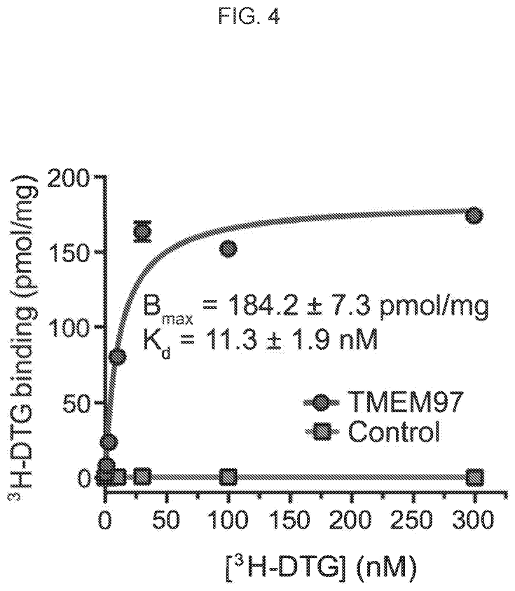

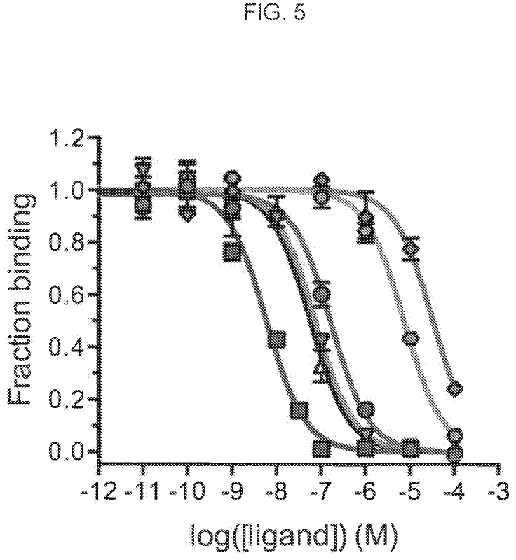

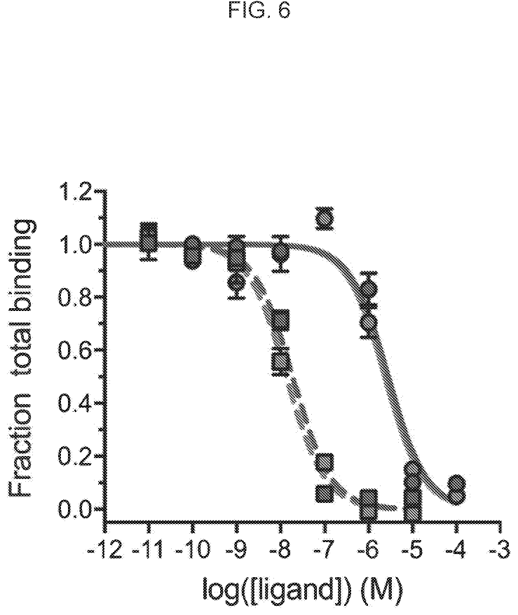

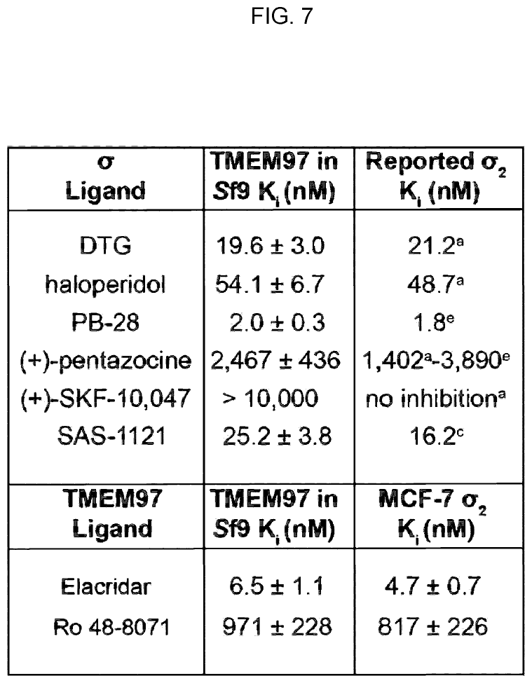

Next, TMEM97 expression was tested for contribution to the sigma-2 binding site. siRNA knockdown was used to reduce mRNA expression of Tmem97 in PC-12 cells by ˜60%. This resulted in a near identical reduction in sigma-2 expression levels as measured by saturating ³H DTG binding (FIG. 3). Next, TMEM97 was characterized to assess whether it possessed the full pharmacological profile of the sigma-2 receptor. TMEM97 was overexpressed in Sf9 insect cells, which lack an endogenous TMEM97 gene and show no significant ³H DTG binding. Expression of TMEM97 resulted in the appearance of saturatable ³H DTG with an affinity of 11.3 nM (FIG. 4), closely in line with literature values ranging from 17.9 to 37.6 nM. Next, competition binding experiments were performed with a collection of sigma-2 ligands representing diverse chemical classes, including DTG, haloperidol, PB-28, (+)-pentazocine, (+)-SKF-10,047, and SAS-1121 (FIG. 5). In each case, the affinity of the ligand for TMEM97 was essentially identical to previously report sigma-2 receptor binding affinities. Two recently reported TMEM97 ligands, Elacridar and Ro 48-8071 were then tested in a similar binding assay. The measured binding affinities of these compounds were identical in both Sf9 membranes from cells overexpressing TMEM97 and in MCF-7 cell membranes, a classical sigma-2 receptor cell line (FIG. 6). Binding affinities for all ligands tested is listed in FIG. 7. Hence, not only do known sigma-2 ligands bind to TMEM97, but also known TMEM97 ligands bind to the sigma-2 receptor. In each case, ligand affinities for the sigma-2 receptor in a classical binding assay are identical to those for recombinant TMEM97. Collectively, these data lead us to conclude that TMEM97 is synonymous with the σ2 receptor.

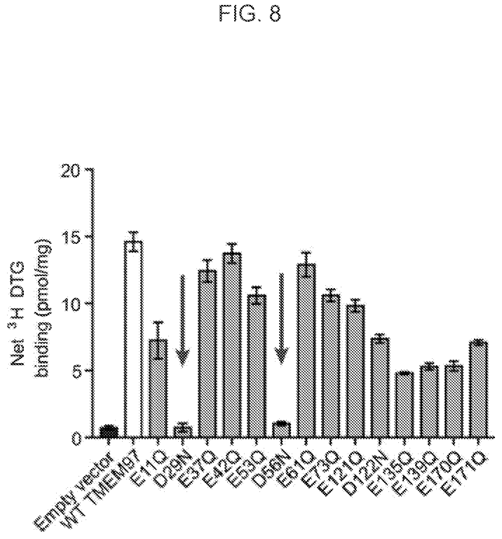

To further characterize the ligand binding site, site-directed mutagenesis was performed on all Glu and Asp residues, hypothesizing that one of these must be the counter-ion to the basic amine found in all sigma-2 ligands. Two mutations, D29N and D56N abolished all binding to ³H DTG (FIG. 8), suggesting that they are essential for ligand binding to sigma-2. This is similar to the sigma-1 receptor, where both Asp126 and Glu172 are required for ligand binding. Furthermore, these residues are in close proximity in a structure prediction model.

### Methods

Purification of Sigma-2 Receptor from Calf Liver

Frozen calf livers (Omaha Steaks) were thawed, cut into 1 cm cubes, and suspended in a buffer of 20 mM HEPES pH 7.5, 2 mM magnesium chloride, and 1:100,000 (v:v) benzonase nuclease (Sigma Aldrich), supplemented with cOmplete Mini, EDTA-free Protease Inhibitor Cocktail Tablets (Roche). Tissue was homogenized with a blender and then centrifuged for 20 minutes at 50,000×g. The supernatant was discarded, and the pelleted membranes were washed by resuspension with a glass dounce tissue grinder in HEPES-buffered saline (20 mM HEPES pH 7.5, 150 mM NaCl). Washing was repeated until protein content in the supernatant was below detection (typically 5-10 washes). Membranes were further washed with HEPES buffered saline supplemented with 2 M urea and then with HEPES-buffered saline supplemented with 0.5 M sodium chloride to remove peripheral membrane proteins.

To extract the receptor, membranes were homogenized with a glass dounce tissue grinder in a 1:5 (v/v) solubilization buffer consisting of 150 mM NaCl, 20 mM HEPES pH 7.5, 10% (v/v) glycerol, and 1% (w/v) lauryl maltose neopentyl glycol (LMNG; Anatrace). Samples were stirred for 2 h at 4° C. and then centrifuged as before for 20 minutes. The resulting supernatant was filtered with a glass microfiber filter (VWR). The filtered supernatant containing solubilized receptor was loaded by gravity flow onto 2 ml affinity resin made by coupling compound JVW-1625 at 100 μM density to Affi-gel 10 (Bio-Rad) according to the manufacturer's instructions. The resin was washed with 50 ml of buffer containing 150 mM NaCl, 20 mM HEPES pH 7.5, 1% glycerol, 0.1% LMNG. The receptor was eluted with 50 ml of the same buffer supplemented with 100 μM 1,3-Di-o-tolylguanidine (DTG) using a syringe pump over a period of three hours and with the eluate flowing directly onto a 250 l hydroxyapatite resin column. Receptor was then eluted from hydroxyapatite resin using 500 l of a buffer containing 500 mM potassium chloride pH 7.2, 25 mM NaCl, 0.1% (w/w) LMNG. Proteins were precipitated by trichloroacetic acid and resolved on SDS-PAGE. A segment of the gel corresponding to molecular weight range 15-25 kDa was sent for liquid chromatography tandem mass spectrometry (LC-MS/MS) analysis at the Harvard Medical School Taplin Mass Spectrometry Facility and at Harvard's Faculty of Arts and Sciences Mass Spectrometry and Proteomics Resource Laboratory.

### Recombinant Receptor Expression

Eight selected hits from LC-MS/MS were cloned into a pTARGET vector (Promega), followed by a porcine teschovirus-1 2A skip peptide (ATNFSLLKQAGDVEENPGP (SEQ ID NO: 3)) and by the fluorescent protein mCardinal40 to assess transfection efficiency. Plasmids were transfected into Expi293 cells (Thermo Fisher) according to manufacturer's instructions. After 36 hours, expression of each target protein was confirmed by flow cytometry analysis of mCardinal fluorescence levels. Receptor point mutants were generated by site-directed mutagenesis using KAPA polymerase (KAPA Biosystems), and resulting constructs were expressed in Expi293 cells as described above.

For insect cell expression, human TMEM97 was cloned into the vector pVL1392, and baculovirus was prepared using the BestBac system (Expression Systems) in accordance with manufacturer's instructions. For large scale production, Sf9 insect cells were infected at a density of 4×106 cells/mL and then shaken at 27° C. for 60 hours prior to harvest.

Preparation of Cell Membranes from Cultured Cells

Membranes were prepared from PC-12, MCF-7, Sf9, or Expi293 cells. In brief, adherent cells were washed with ice cold PBS or HBS and harvested with a cell scraper, while suspension cells were simply pelleted. Cell pellets were suspended in 20 mM HEPES pH 7.5, 2 mM magnesium chloride, and 1:100,000 (v:v) benzonase nuclease (Sigma Aldrich) and supplemented with cOmplete Mini, EDTA-free Protease Inhibitor Cocktail Tablets (Roche). Following dounce homogenization, the cells were centrifuged at 50,000×g for 20 minutes. The supernatant was discarded and the membranes were washed one more time with cold 50 mM Tris pH 8.0 containing cOmplete Mini, EDTA-free Protease Inhibitor Cocktail Tablets (Roche, 1 tablet per 50 mL buffer). The membranes were centrifuged once more at 50,000× for 20 minutes, and then resuspended in a variable volume of cold 50 mM Tris pH 8.0 with the same protease inhibitor cocktail. Protein content was assessed by DC protein assay (Bio-Rad) according to manufacturer's instructions. Membranes were aliquoted, flash frozen, and stored at −80° C. until use in the radioligand binding experiments described below.

### Single-Point Radioligand Binding Assays

Membrane radioligand binding assays were performed as described 41 with slight modifications. Briefly, samples were incubated with 10-30 nM ³H 1,3-di-o-tolylguanidine (DTG; Perkin Elmer) in 50 mM Tris pH 8 buffer and supplemented with either 1.8 μM (+)SKF-10,047 or 50 nM PD-144418, both potent and selective σ1 receptor ligands, to block sigma-1 sites. Nonspecific binding was measured by the addition of 2 μM haloperidol to otherwise identical conditions measured in parallel. For siRNA experiments in PC-12 cells, 30 nM ³H DTG was isotopically diluted with 270 nM cold DTG to ensure total σ2 binding was assayed. Samples were incubated at room temperature with shaking for 1.5 hours, at which time the reaction was terminated by the addition of ice-cold water. Samples were then applied to glass fiber filters (Merck Millipore) that had been pre-treated with 0.3% (v/v) polyethylenimine. Filters were immediately washed twice with ice-cold water and then dried. Radioactivity was measured by liquid scintillation counting.

Radioligand binding experiments on extracted samples were done with slight modifications. Incubation with ³H DTG was done without shaking. Bound radioligand was separated from unbound using a desalting column of G50 fine resin (GE Healthcare).

### ³H DTG Saturation Binding in Cell Membranes

³H DTG saturation binding to membranes was determined using an assay where membranes from infected Sf9 insect cells (2.5 μg total protein per reaction) or MCF-7 cells (15-30 μg total protein per reaction) were incubated in a 100 μL reaction buffered with 50 mM Tris pH 8.0, containing 1.8 μM (+)SKF-10,047, and 0-30 nM ³H DTG. Concentrations of 100 and 300 nM DTG were assayed by isotopic dilution to minimize use of ³H DTG. For each membrane type, a second curve that was otherwise identical save for the addition of 2 μM haloperidol was measured in parallel to determine nonspecific binding. Reactions were incubated at 37° C. for 90 minutes and then terminated via filtration through glass fiber filter using a Brandel cell harvester. After washing, filters were soaked in 5 mL Cytoscint scintillation fluid overnight and measured on a Beckman Coulter LS 6500 scintillation counter. Kd values were calculated using non-linear regression tools from Graphpad Prism.

### Competition Binding Assays in Cell Membranes

³H DTG competition curves testing the binding of 6 ligands haloperidol, DTG, PB-28, SAS-1121, (+)-pentazocine, and (+)-SKF-10,047, or the TMEM97 ligands Elacridar or Ro 48-8071, were performed. Briefly, Sf9 insect membranes overexpressing TMEM97 (2.5 μg of total protein per reaction) or MCF-7 membranes (12-30 μg total protein per reaction) were incubated in a 100 μL reaction buffered with 50 mM Tris pH 8.0, with 30 nM ³H DTG and eight concentrations ranging from 10 pM-100 μM of the competing cold ligand. 1.8 μM (+)SKF-10,047 was included to block 1l receptor sites in all MCF-7 membranes binding assays and in Sf9 membranes when testing TMEM97 ligands. Reactions were incubated for 90 minutes at 37° C., and were then terminated by filtration through a glass fiber filter using a Brandel cell harvester. Glass fiber filters were soaked in 0.3% polyethylenimine for at least 30 minutes at room temperature prior to harvesting. All reactions were performed in triplicate using a 96-well block. After the membranes were transferred to the filters and washed, the filters were soaked in 5 mL Cytoscint scintillation fluid overnight and radioactivity was measured using a Beckman Coulter LS 6500 scintillation counter. Data were analyzed using software from Graphpad Prism. Kᵢ values were computed directly without the use of the Cheng-Prusoff correction using the Graphpad Prism software.

siRV Knockdown of TMEM97

A pair of siRNA oligos were designed against Rattus norvegicus Tmem97 mRNA using the Stealth RNAi™ tool available through ThermoFisher Scientific. The sense strand for the siRNA was 5′-CAACCUGUUGCGGUGGUACUCUAAG-3′ (SEQ ID NO: 4), and the antisense strand was 5′-CUUAGAGUACCACCGCAACAGGUUG-3′ (SEQ ID NO: 5). As a control, we used the AllStars Negative Control siRNA™ from Qiagen. For the transfection, 2.2×106 PC-12 cells suspended in 8.0 mL of DMEM with 10% FBS and 10 μg/mL of gentamicin were placed in a 10 cm dish and immediately transfected with a 2.0 mL solution containing 20 μL of Lipofectamine® RNAimax from ThermoFisher Scientific and 10 nM of either the control or Tmem97 siRNA. After 24 hours, the media was changed for fresh media, and the cells were transfected again in the same way. 48 hours after the first transfection, the cells were trypsinized and split 1:2 into two new 10 cm plates. On the 5th day after the first transfection, cells were harvested by trypsinization and centrifugation, and 10% were set aside for RNA extraction while 90% were used for binding analysis.

RNA extraction was done using the RNeasy® kit from Qiagen according to the manufacturer's instructions. The RNA was converted to cDNA using Invitrogen's SuperScript® II reverse transcriptase kit according to the manufacturer's instructions. RNA was removed from the cDNA after reverse transcription by digestion using E. coli RNase H.

### Real-Time Quantitative PCR for Quantification of TMEM97 mRA Levels in PC-12 Cells

After preparation of the cDNA was complete, real-time quantitative PCR (qPCR) was performed on a QuantStudio 6 qPCR instrument at the Harvard Medical School Center for Molecular Interactions using PowerUp™ SYBR® Green Master Mix from Applied Biosystems Life Technologies. The qPCR was performed according to the master mix manufacturer recommendations, using a range of different template input concentrations and final primer concentration of 250 nM. Primers for qPCR were designed using the NCBI primer design tools. The primers used for the forward and reverse primer for Rattus norvegicus Tmem97 were 5′-TACTTCGTCTCGCACATCCC-3′ (SEQ ID NO: 6) and 5′-TTGCTGAACTCCTGCGGGTA-3′ (SEQ ID NO: 7) respectively. Rattus norvegicus actb was used as a reference gene, for which the forward and reverse primers were 5′-CCCGCGAGTACAACCTTCTTG-3′ (SEQ ID NO: 8) and 5′-GTCATCCATGGCGAACTGGTG-3′ (SEQ ID NO: 9) respectively. Fold differences in Tmem97 expression levels were calculated using the ΔΔC_{T} method 43. All measurements were performed in triplicate.

### General Synthetic Chemistry Methods

Methylene chloride (CH₂Cl₂), N,N-diisopropylethylamine (i-Pr₂NEt), triethylamine (Et₃N), tert-butanol (tBuOH), and ethanol (EtOH) were distilled from calcium hydride (CaH2) immediately prior to use. All reagents were reagent grade and used without purification unless otherwise noted. Where required, solvents were degassed by sparging with nitrogen prior to use. All reactions involving air or moisture sensitive reagents or intermediates were performed under an inert atmosphere of nitrogen or argon in glassware that was flame dried. Reaction temperatures refer to the temperature of the cooling/heating bath. Volatile solvents were removed under reduced pressure using a Büchi rotary evaporator. Thin-layer chromatography (TLC) was performed on EMD 60 F254 glass-backed pre-coated silica gel plates and were visualized using one or more of the following methods: UV light (254 nm) and staining with basic potassium permanganate (KMnO₄) or acidic p-anisaldehyde (PAA). Proton nuclear magnetic resonance (¹H NMR) and carbon nuclear magnetic resonance (¹³C NMR) spectra were obtained at the indicated field as solutions in CDCl₃ unless otherwise indicated. Chemical shifts are referenced to the deuterated solvent and are reported in parts per million (ppm, δ) downfield from tetramethylsilane (TMS, δ=0.00 ppm). Coupling constants (J) are reported in Hz and the splitting abbreviations used are: s, singlet; d, doublet; t, triplet; q, quartet; m, multiplet; comp, overlapping multiplets of magnetically nonequivalent protons; br, broad; app, apparent.

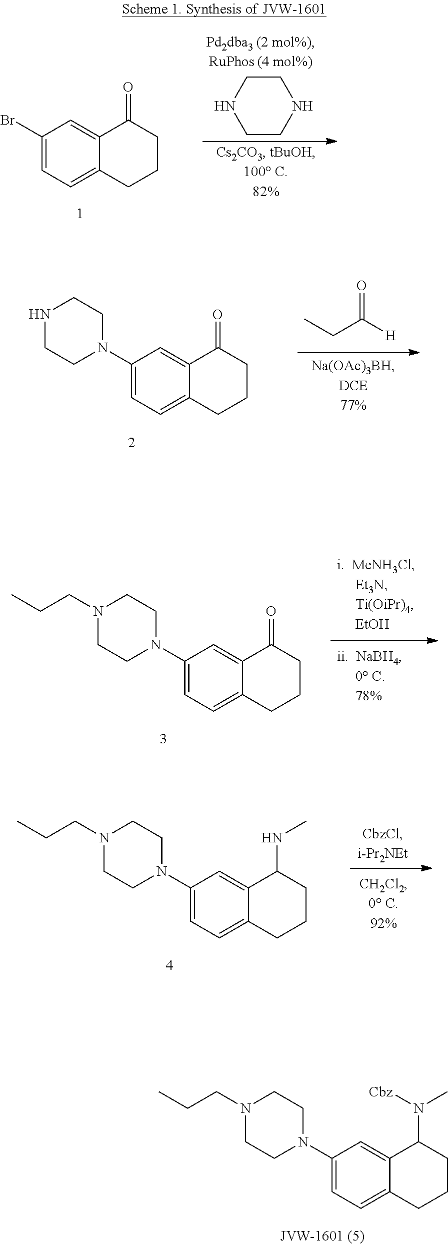

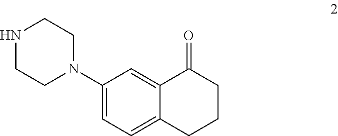 [SMILES: O=C1CCCc2ccc(N3CCNCC3)cc21]

### 7-(Piperazin-1-yl)-3,4-dihydronaphthalen-1(2H)-one (2)

A resealable tube was charged with tetralone 1 (0.500 g, 2.22 mmol), piperazine (1.913 g, 22.2 mmol), Cs₂CO₃ (1.086 g, 3.33 mmol) and degassed tBuOH (11.1 mL). The suspension was stirred at 45° C. for 15 min, whereupon a freshly prepared tBuOH solution (0.67 mL) containing Pd₂dba₃ (40.6 mg, 0.044 mmol) and RuPhos (41.5 mg, 0.088 mmol) that had been stirred at 60° C. for 30 min was added. The tube was sealed, and the reaction was stirred at 100° C. for 3 h. After cooling to room temperature, the mixture was filtered through Celite®, the filter cake was washed with CH₂Cl₂ (200 mL), and the filtrate was concentrated. The residue was dissolved in CH₂Cl₂ (50 mL), washed with saturated aq. NaHCO₃ (2×50 mL), and extracted with 1 N HCl (4×30 mL). The combined acidic aqueous extracts were made basic with 6 N NaOH and extracted with CH₂Cl₂ (4×50 mL), after which the combined organic extracts were dried (Na₂SO₄) and concentrated under reduced pressure. The crude material was purified via flash chromatography (SiO₂) eluting with CH₂Cl₂/MeOH/Et₃N (97:2:1) affording 0.418 g (82%) of 2 as a red oil. ¹H NMR (400 MHz, CDCl₃) δ 7.46 (d, J=2.8 Hz, 1H), 7.08 (d, J=8.4 Hz, 1H), 7.02 (dd, J=8.4, 2.8 Hz, 1H), 3.14-3.06 (comp, 4H), 3.00-2.93 (comp, 4H), 2.79 (t, J=6.1 Hz, 2H), 2.70 (br s, 1H), 2.54 (m, 2H), 2.01 (m, 2H); ¹³C NMR (101 MHz, CDCl₃) δ 198.5, 150.3, 135.8, 132.8, 129.4, 122.0, 112.8, 50.0, 45.8, 39.1, 28.7, 23.4; HRMS (ESI) m/z calcd for C₁₄H₁₈N₂O (M+H)⁺, 231.1492; found 231.1497

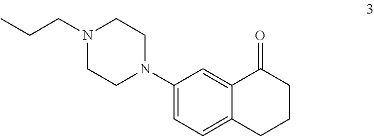 [SMILES: CCCN1CCN(c2ccc3c(c2)C(=O)CCC3)CC1]

### 7-(4-Propylpiperazin-1-yl)-3,4-dihydronaphthalen-1(2H)-one (3)

A solution of propionaldehyde (1.006 g, 11.29 mmol) in dichloroethane (25 mL) was added dropwise to a solution of amine 2 (2.363 g, 10.3 mmol) and Na(OAc)₃BH (4.349 g, 20.5 mmol) in DCE (103 mL), and the reaction was stirred at room temperature for 3 h. The reaction mixture was then washed with saturated aq. NaHCO₃ (2×50 mL), dried (Na₂SO₄), and concentrated under reduced pressure. The crude material was purified via flash chromatography (SiO₂) eluting with hexanes/EtOAc/Et₃N (74:25:1) affording 2.165 g (77%) of 3 as a yellow oil. ¹H NMR (400 MHz, CDCl₃) δ 7.54 (d, J=2.7 Hz, 1H), 7.14 (d, J=8.4 Hz, 1H), 7.08 (dd, J=8.5, 2.7 Hz, 1H), 3.24-3.19 (comp, 4H), 2.86 (t, J=6.1 Hz, 2H), 2.64-2.55 (comp, 6H), 2.38-2.31 (m, 2H), 2.09 (m, 2H), 1.60-1.48 (m, 2H), 0.92 (t, J=7.4 Hz, 3H); ¹³C NMR (101 MHz, CDCl₃) δ 198.9, 150.1, 135.8, 133.0, 129.6, 122.0, 113.0, 60.8, 53.2, 49.1, 39.3, 28.9, 23.6, 20.1, 12.1; HRMS (ESI) m/z calcd for C₁₇H₂₄N₂O (M+H)⁺, 273.1961; found 273.1965

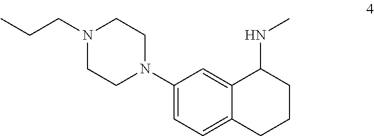 [SMILES: CCCN1CCN(c2ccc3c(c2)C(NC)CCC3)CC1]

### N-Methyl-7-(4-propylpiperazin-1-yl)-1,2,3,4-tetrahydronaphthalen-1-amine (4)

Tetralone 3 (0.102 g, 0.375 mmol) was dissolved in EtOH (2.5 mL) in a resealable tube, whereupon Ti(OiPr)₄ (1.44 g, 1.5 mL, 3.75 mmol), Et₃N (0.19 g, 0.26 mL, 1.9 mmol) and MeNH₃Cl (0.127 g, 1.88 mmol) were sequentially added. The tube was sealed, and the reaction was stirred at room temperature for 7 h. The solution was cooled to 0° C., and NaBH₄ (0.028 g, 0.75 mmol) was added in one portion. Stirring was continued at 0° C. for 1 h, and the mixture was added to 2 M aq. NH₄OH (10 mL). The suspension was filtered through a pad of Celite®, and the filter cake was washed with hot EtOAc (150 mL). The filtrate was concentrated under reduced pressure and partitioned between CH₂Cl₂ (15 mL) and saturated aq. NaHCO₃ (10 mL). The organic layer was separated and extracted with 1 M HCl (3×15 mL). The combined aqueous extracts were adjusted to pH 10 with 6 M NaOH and extracted with CH₂Cl₂ (3×50 mL). The combined organic extracts were dried (Na₂SO₄) and concentrated under reduced pressure. The crude residue was purified via flash chromatography (SiO₂) eluting with CH₂Cl₂/MeOH/Et₃N (98:1:1) affording 0.0763 g (78%) of 4 as a pale yellow oil. ¹H NMR (400 MHz, CDCl₃) δ 6.97 (d, J=8.4 Hz, 1H), 6.92 (d, J=2.6 Hz, 1H), 6.77 (dd, J=8.4, 2.7 Hz, 1H), 3.61 (t, J=4.9 Hz, 1H), 3.20-3.14 (comp, 4H), 2.77-2.62 (comp, 2H), 2.62-2.57 (comp, 4H), 2.49 (s, 3H), 2.38-2.32 (comp, 2H), 1.96-1.81 (comp, 3H), 1.75-1.66 (m, 1H), 1.60-1.49 (comp, 2H), 1.37 (br s, 1H), 0.92 (t, J=7.4 Hz, 3H); ¹³C NMR (101 MHz, CDCl₃) δ 149.7, 139.6, 129.7, 128.8, 116.4, 115.4, 60.8, 57.6, 53.4, 49.8, 34.1, 28.6, 27.9, 20.1, 19.2, 12.1; HRMS (ESI) m/z calcd for C₁₈H₂₉N₃ (M+H)⁺, 288.2434; found 288.2438

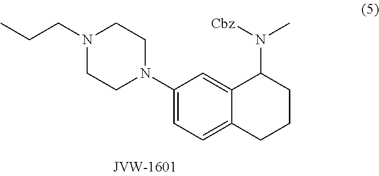 [SMILES: CCCN1CCN(c2ccc3c(c2)C(N(C)C(=O)OCc2ccccc2)CCC3)CC1]

### Benzyl methyl(7-(4-propylpiperazin-1-yl)-1,2,3,4-tetrahydronaphthalen-1-yl)carbamate (5)

i-Pr₂NEt (0.0519 g, 70 μL, 0.377 mmol) and CbzCl (0.0478 g, 40 μL, 0.276 mmol) were added with stirring to a solution of amine 4 (0.0721 g, 0.251 mmol) in CH₂Cl₂ (1.3 mL) cooled to 0° C. The solution was stirred at 0° C. for 4 h and then diluted with CH₂Cl₂ (10 mL). The solution was washed with 1 N HCl (2×10 mL), 1 N NaOH (2×10 mL), saturated aqueous NaHCO₃ (1×10 mL), dried (Na₂SO₄), and concentrated under reduced pressure. The crude residue was purified via flash chromatography (SiO₂) eluting with hexanes/EtOAc/Et₃N (74:25:1) affording 0.0969 (92%) g of 5 as a colorless oil. ¹H NMR (499 MHz, CDCl₃) (mixture of rotamers) δ 7.45-7.27 (comp, 5H), 7.02-6.96 (m, 1H), 6.81-6.75 (m, 1H), 6.66-6.60 (m, 1H), 5.51-5.11 (comp, 3H), 3.16-3.05 (comp, 4H), 2.75-2.61 (comp, 5H), 2.61-2.54 (comp, 4H), 2.39-2.33 (comp, 2H), 2.08-1.92 (comp, 2H), 1.83-1.68 (comp, 2H), 1.61-1.51 (comp, 2H), 0.94 (t, J=7.4 Hz, 3H); ¹³C NMR (126 MHz, CDCl₃) (mixture of rotamers) δ 157.2, 157.0, 150.1, 150.0, 137.2, 137.1, 135.8, 135.7, 130.0, 129.9, 129.8, 129.6, 128.5, 127.9, 127.8, 127.8, 115.6, 115.4, 114.6, 114.2, 67.2, 67.1, 60.8, 55.6, 55.4, 53.3, 53.3, 49.6, 49.5, 30.3, 29.7, 28.8, 28.7, 28.2, 27.7, 22.3, 22.1, 20.1, 12.1; HRMS (ESI) m/z calcd for C₂₆H₃₅N₃O₂ (M+H)⁺, 422.2802; found 422.2816

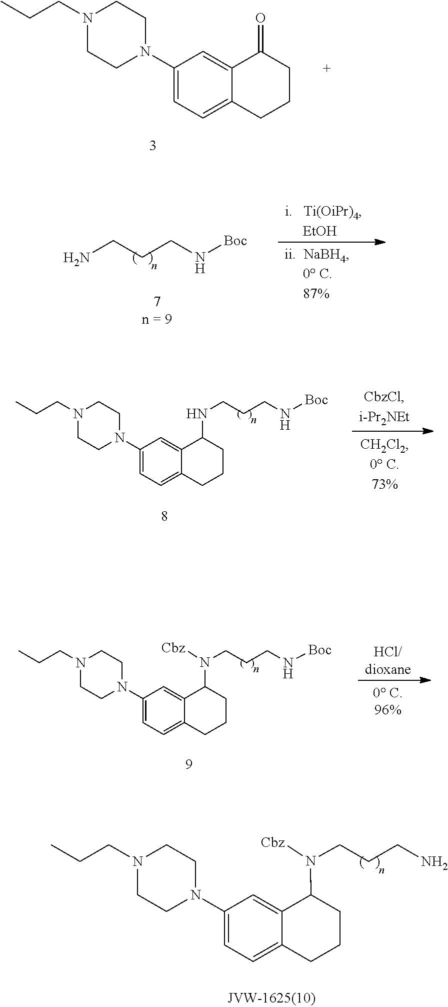

### Scheme 2. Synthesis of JVW-1625 (10)

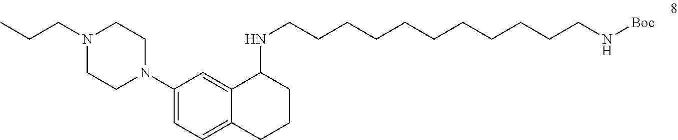 [SMILES: CCCN1CCN(c2ccc3c(c2)C(NCCCCCCCCCCCNC(=O)OC(C)(C)C)CCC3)CC1]

### tert-Butyl (11-((7-(4-propylpiperazin-1-yl)-1,2,3,4-tetrahydronaphthalen-1-yl)amino)undecyl)carbamate (8)

Tetralone 3 (0.0508 g, 0.186 mmol) was dissolved in EtOH (1.2 mL) in a screw cap vial, whereupon the known amine 7 (0.0801 g, 0.279 mmol) and Ti(OiPr)₄ (0.5 g, 0.6 mL, 1.9 mmol) were sequentially added. The vial was sealed, and the reaction was stirred at 45° C. for 22 h. The solution was cooled to 0° C. and NaBH₄ (0.014 g, 0.37 mmol) was added in one portion. Stirring was continued at 0° C. for 1 h, and the mixture was added to 2 M aq. NH₄OH (10 mL). The suspension was filtered through a pad of Celite®, and the filter cake was washed with hot EtOAc (250 mL). The filtrate was concentrated under reduced pressure and partitioned between CH₂Cl₂ (20 mL) and 1 M NaOH (15 mL). The organic layer was separated, and the aqueous layer was extracted with CH₂Cl₂ (2×15). The combined organic extracts were dried (Na₂SO₄) and concentrated under reduced pressure. The residue was purified via flash chromatography (SiO₂) eluting with hexanes/acetone/Et₃N (84:15:1) afforded 0.0876 g (87%) of 8 as a colorless oil. ¹H NMR (499 MHz, CDCl₃) δ 6.96 (d, J=8.4 Hz, 1H), 6.93 (d, J=2.6 Hz, 1H), 6.76 (dd, J=8.4, 2.6 Hz, 1H), 4.56 (br s, 1H), 3.69 (t, J=5.0 Hz, 1H), 3.20-3.13 (m, 4H), 3.08 (comp, J=6.8 Hz, 2H), 2.75 547-2.62 (comp, 4H), 2.61-548 2.57 (comp, 4H), 2.37-2.32 (comp, 2H), 1.97-1.87 (m, 1H), 1.86-1.79 (comp, 2H), 1.72-549 1.64 (m, 1H), 1.60-1.40 (comp, 15H), 1.38-1.22 (m, 15H), 0.92 (t, J=7.4 Hz, 3H); ¹³C 550 NMR (126 MHz, CDCl₃) δ 156.1, 149.7, 140.1, 129.7, 129.0, 116.4, 115.5, 79.1, 60.9, 56.0, 551 53.4, 49.9, 47.4, 40.7, 30.7, 30.2, 29.7, 29.7, 29.7, 29.6, 29.4, 28.7, 28.6, 28.5, 27.6, 26.9, 20.2, 552 19.4, 12.1; HRMS (ESI) m/z calcd for C₃₃H₅₈N₄O₂ (M+H)⁺, 543.4633; found 543.4651

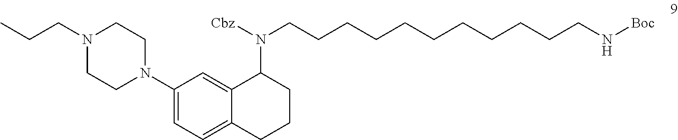 [SMILES: CCCN1CCN(c2ccc3c(c2)C(N(CCCCCCCCCCCNC(=O)OC(C)(C)C)C(=O)OCc2ccccc2)CCC3)CC1]

### Benzyl (1-((tert-butoxycarbonyl)amino)undecyl)(7-(4-propylpiperazin-1-yl)-1,2,3,4-tetrahydronaphthalen-1-yl)carbamate (9)

i-Pr₂NEt (0.038 g, 52 μL, 0.30 mmol) and CbzCl (0.038 g, 32 μL, 0.22 mmol) were added with stirring to a solution of amine 8 (0.0804 g, 0.148 mmol) in CH₂Cl₂ (0.75 mL) cooled to 0° C. The solution was stirred at 0° C. for 1 h and then diluted with CH₂Cl₂ (20 mL). The solution was washed with 1 N HCl (2×10 mL), 1 N NaOH (2×20 mL), saturated aqueous NaHCO₃ (1×20 mL), dried (Na₂SO₄), and concentrated under reduced pressure. The crude residue was purified via flash chromatography (SiO₂) eluting with hexanes/EtOAc/Et₃N (74:25:1) affording 0.0806 g (80%) of 9 as a pale yellow oil. ¹H NMR (499 MHz, CDCl₃) (mixture of rotamers) δ 7.43-7.19 (comp, 5H), 6.99-6.95 (m, 1H), 6.76 (dd, J=8.4, 2.5 Hz, 1H), 6.60 (d, J=2.5 Hz, 1H), 5.42-4.98 (m, 3H), 4.51 (br s, 1H), 3.29-3.01 (comp, 7H), 2.88-2.50 (comp, 7H), 2.41-2.31 (comp, 2H), 2.10-1.38 (comp, 19H), 1.32-1.07 (comp, 14H) 0.93 (t, J=7.4 3H); ¹³C NMR (126 MHz, CDCl₃) (mixture of rotamers) δ 157.3, 156.6, 156.1, 150.0, 149.9, 137.3, 137.2, 137.1, 136.7, 130.0, 129.8, 129.5, 128.6, 128.5, 128.0, 127.9, 127.9, 115.7, 115.4, 114.5, 114.0, 79.1, 67.1, 67.0, 60.8, 56.5, 53.3, 49.6, 49.5, 45.0, 40.8, 30.6, 30.5, 30.2, 29.6, 29.6, 29.5, 29.4, 29.3, 29.3, 28.9, 28.8, 28.6, 27.3, 26.9, 22.7, 22.5, 20.2, 12.1; HRMS (ESI) m/z calcd for C₄₁H₆₄N₄O₄ (M+H)⁺, 677.5000; found 677.4994

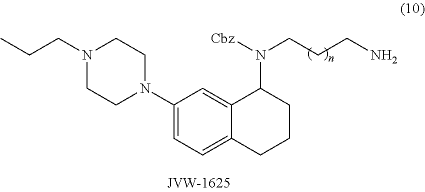

### Benzyl (11-aminoundecyl)(7-(4-propylpiperazin-1-yl)-1,2,3,4-tetrahydronapthalen-1-yl)carbamate (10)

Carbamate 9 (0.0373 g, 0.0551 mmol) was dissolved in a solution of dioxane (0.4 mL) containing HCl (4 M) cooled to 0° C. The solution was stirred at 0° C. for 3 h and then diluted with CH₂Cl₂ (20 mL). The solution was washed with 1 N NaOH (1×20 mL), saturated aqueous NaHCO₃ (1×20 mL), dried (Na₂SO₄), and concentrated under reduced pressure to afford 0.0306 g of 10 (96%) as a pale yellow oil. ¹H NMR (499 MHz, CDCl₃) (mixture of rotamers) δ 7.44-7.18 (comp, 5H), 7.01-6.94 (m, 1H), 6.76 (dd, J=8.4, 2.5 Hz, 1H), 6.63-6.56 (m, 1H), 5.44-5.01 (comp, 3H), 3.29-2.99 (comp, 5H), 2.86-2.60 (comp, 5H), 2.58-2.50 (comp, 4H), 2.38-2.31 (comp, 2H), 2.07-1.41 (comp, 12H), 1.32-1.08 (comp, 14H), 0.93 (t, J=7.4 Hz, 3H); ¹³C NMR (126 MHz, CDCl₃) (mixture of rotamers) δ 157.2, 156.5, 149.8, 149.7, 137.2, 136.9, 136.5, 129.8, 129.7, 129.3, 128.4, 128.4, 127.8, 127.8, 127.7, 115.6, 115.2, 114.3, 113.8, 66.9, 66.8, 60.7, 56.3, 53.2, 53.2, 49.5, 44.8, 42.1, 33.5, 30.4, 29.6, 29.5, 29.5, 29.4, 29.2, 29.1, 28.7, 28.6, 27.2, 26.9, 22.6, 22.4, 20.1, 12.0; HRMS (ESI) m/z calcd for C₃₆H₅₆N₄O₂ (M+H)⁺, 577.4476; found 577.4484

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A method of treating a subject having a sterol homeostasis disease, the method comprising administering to the subject a sigma-2 receptor ligand in an amount and for a duration sufficient to treat the sterol homeostasis disease.

2. The method of claim 1, wherein the ligand is a sigma-2 receptor agonist, antagonist, or partial agonist.

3. The method of claim 2, wherein the ligand is selected from the group consisting of: opipramol, MIN-101 (2-[[1-[2-(4-fluorophenyl)-2-oxoethyl]piperidin-4-yl]methyl]-3H-isoindol-1-one), CT-1812, siramesine, rimcazole, ibogaine, afobazole, BMY-14802 (1-(4-fluorophenyl)-4-[4-(5-fluoro-2-pyrimidinyl)-1-piperazinyl]-1-butanol), and panamesine.

4. The method of claim 2, wherein the ligand is selected from the group consisting of: ¹¹C-PB-28, ¹²⁵I RHM-4, ¹²⁵I-IAC44, ¹²⁵I-IAF(1-N-(2′,6′-dimethyl-morpholino)-3-(4-azido-3-[(125)I]iodo-phenyl)propane, ¹⁸F ISO-1, 2-(4-(3-(4-fluorophenyl)indol-1-yl)butyl)-6,7-dimethoxy-1,2,3,4-tetrahydroisoquinoline), ³H DTG, ³H-azido-DTG, ³H-PB28, ³H-RHM-1, ⁹⁹mTc BAT-EN6, ^{99m}Tc-4-(4-cyclohexylpiperazine-1-yl)-butan-1-one-1-cyclopentadienyltricarbonyl technetium, ABN-1, AG-205, ANSTO-19, benzoxazolone, BIMU-1, CB-182, CB-184, CB-64D, CB-64L, cocaine, ditolylguanidine (DTG), F281, indole ((1-[3-[4-(substituted-phenyl) piperazin-1-yl]-propyl]-1H-indole, K05-I38, K05-I38, N-Benzyl-7-azabicyclo[2.2.1]heptane, PB 183, PB28, RHM-1, RHM-138, RHM-2, RHM-4, SM-21, SN79, SV119, SW107, SW116, SW120, SW43, TC4ANSTO-19, WC-21, WC-26, WC-59, yun179, yun194, yun201, yun202, yun203, yun204, yun209, yun210, yun212, yun234, yun236, yun242, yun243 (RMH-1), yun245, yun250, yun251, yun253, yun254, yun552, SAS-0132, DKR-1051, DKR-1005, JVW-1009, and SAS-1121.

5. The method of claim 2, wherein the ligand is a compound having the formula: 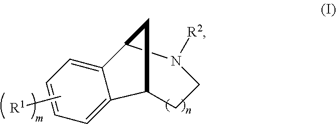
wherein:
R¹ is hydrogen, halogen (e.g., —Cl), —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —N₃, —CF₃, —CCl₃, —CBr₃, —CCI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n1 and n2 are independently 1 or 2;
m is 1, 2, 3 or 4;
n is 1 or 2; and
R³, R^{3A}, R⁴, R^{4A} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

6. The method of claim 5, wherein the ligand is a compound having the formula: 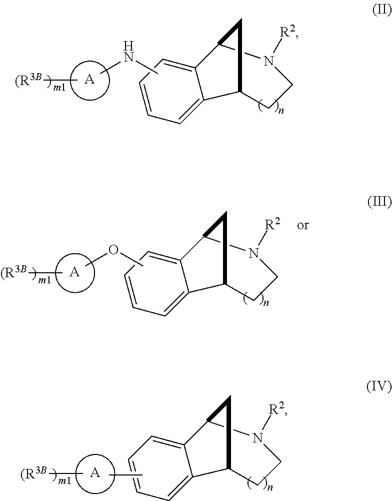
wherein
R^{3B} is —CF₃, —CN, —OH, —NH₂, —CONH₂, —S(O)₃H, —S(O)₂NH₂, —NHC(O) NH₂, —NHC(O)H, —OCHF₂, oxo, halogen, —COOH, —NO₂, —SH, —S(O)₄H, —NHNH₂, —ONH₂, —NHC(O)NHNH₂, —NHS(O)₂H, —NHC(O)—OH, —NHOH, —OCF₃, unsubstituted alkyl, unsubstituted heteroalkyl, unsubstituted cycloalkyl, unsubstituted heterocycloalkyl, unsubstituted aryl or unsubstituted heteroaryl;
ring A is aryl, heteroaryl, cycloalkyl or heterocycloalkyl; and
m1 is 0, 1, 2, 3, or 4.

7. The method of claim 6, wherein the ligand is a compound having the formula:

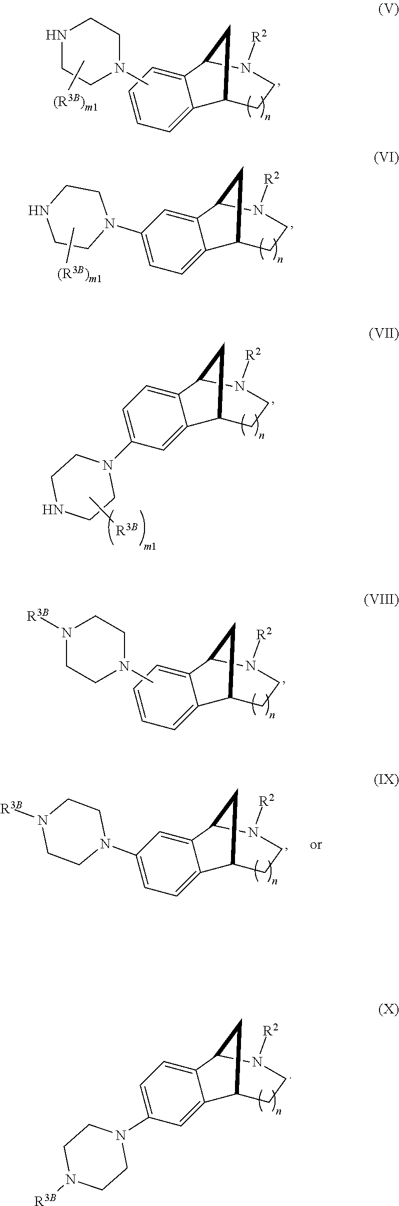

8. The method of claim 2, wherein the ligand is a compound having the formula: 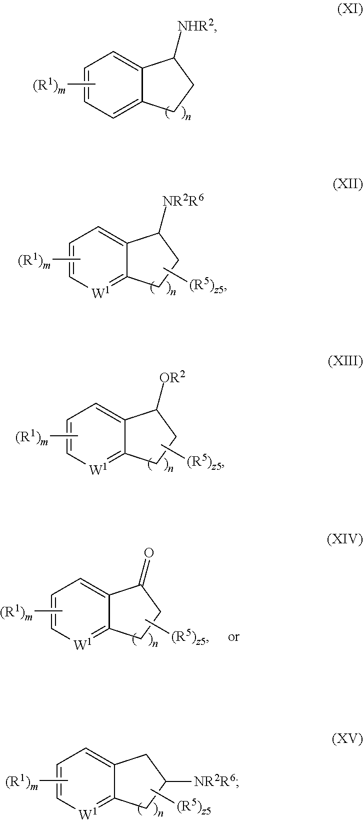
wherein R¹ is hydrogen, halogen (e.g., —F, —Cl, —Br, —I), —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, substituted or unsubstituted alkyl, substituted or unsubstituted heteroalkyl, substituted or unsubstituted cycloalkyl, substituted or unsubstituted heterocycloalkyl (e.g., piperazinyl, piperidinyl, morpholinyl), substituted or unsubstituted aryl (e.g., phenyl), or substituted or unsubstituted heteroaryl (e.g., pyridyl); R² is hydrogen, halogen, —N₃, —CF₃, —CCl₃, —CBr₃, —CI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, substituted or unsubstituted alkyl
(e.g., —CH₃, —CH₂CH₃, —CH₂CH₂CH₃, —CH₂CH₂CH₂OH, —CH₂Ph), substituted or unsubstituted heteroalkyl
(e.g., —C(O)OCH₂Ph, —C(O)NHCH₂Ph, —CH₂CH₂C(O)OCH₂CH₃, —CH₂CH₂C(O)OCH₃, —CH₂CH₂OCH₂CH₃, —CH₂CH₂OCH₃), substituted or unsubstituted cycloalkyl, substituted or unsubstituted heterocycloalkyl (e.g., tetrahydropyranyl, piperidinyl, methyl substituted piperidinyl), substituted or unsubstituted aryl, or substituted or unsubstituted heteroaryl; the symbols n1 and n2 are independently 1 or 2; the symbol m is 1, 2, 3 or 4; n is 1, 2, 3 or 4; R³, R^{3A}, R⁴, R^{4A} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O)₂NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is halogen, —N₃, —CF₃, —CCl₃, —CBr₃, —CI₃, —CN, —C(O)R^{5C}, —OR^{5D} (e.g., —OH), —NR^{5A}R^{5B}, —C(O)OR^{5D}, —C(O)NR^{5A}R^{5B}, —NO₂, —SR^{5D}, —S(O)ₙ₅R^{5C}, —S(O)ₙ₅OR^{5D}, —S(O)ₙ₅NR^{5A}R^{5B}, —NHNR^{5A}R^{5B}, —ONR^{5A}R^{5B}, —NHC(O)NHNR^{5A}R^{5B}, substituted or unsubstituted alkyl (e.g., —CH₂Ph), substituted or unsubstituted heteroalkyl, substituted or unsubstituted cycloalkyl, substituted or unsubstituted heterocycloalkyl, substituted or unsubstituted aryl, or substituted or unsubstituted heteroaryl; the symbol n5 is independently 1 or 2; the symbol z5 is independently an integer from 0 to 6; R⁶ is halogen, —N₃, —CF₃, —CCl₃, —CBr₃, —CI₃, —CN, —C(O)R^{6C}, —OR^{6D}, —NR^{6A}R^{6B}, —C(O)OR^{6D}, —C(O)NR^{6A}R^{6B}, —NO₂, —SR^{6D}, —S(O)ₙ₆R^{6C}, —S(O)ₙ₆OR^{6D}, —S(O)ₙ₆NR^{6A}R^{6B}, —NHNR^{6A}R^{6B}, —ONR^{6A}R^{6B}, —NHC(O)NHNR^{6A}R^{6B}, substituted or unsubstituted alkyl
(e.g., —CH₃, —CH₂CH₃, —CH₂CH₂CH₃, —CH₂CH₂CH₂OH, —CH₂Ph), substituted or unsubstituted heteroalkyl
(e.g., —C(O)OCH₂Ph, —CH₂CH₂C(O)OCH₂CH₃, —CH₂CH₂C(O)OCH₃, —CH₂CH₂OCH₂CH₃, —CH₂CH₂OCH₃), substituted or unsubstituted cycloalkyl, substituted or unsubstituted heterocycloalkyl (e.g., tetrahydropyranyl, piperidinyl, methyl substituted piperidinyl), substituted or unsubstituted aryl, or substituted or unsubstituted heteroaryl; the symbol n6 is independently 1 or 2; W¹ is CH, C(R¹), or N; and R^{5A}, R^{5B}, R^{5C}, R^{5D}, R^{6A}, R^{6B}, R^{6C}, and R^{6D} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

9. The method of claim 2, wherein the ligand is a compound having the formula:

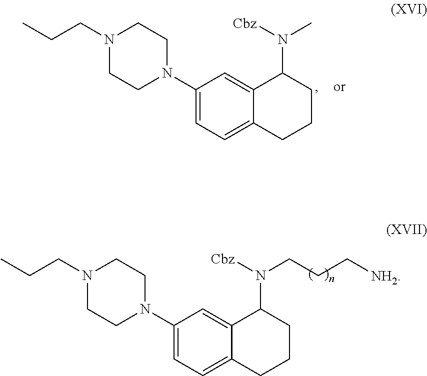

10. The method of any one of claims 1 to 9, wherein the sterol homeostasis disease is Niemann-Pick disease.

11. The method of claim 10, wherein the Niemann-Pick disease is Niemann-Pick type C disease.

12. The method of claim 11, wherein the Niemann-Pick type C disease is Niemann-Pick type C1 disease.

13. A method of treating a subject having a neurological condition, the method comprising administering to the subject a TMEM97 ligand in an amount and for a duration sufficient to treat the neurological condition.

14. The method of claim 13, wherein the ligand is a TMEM97 agonist, antagonist, or partial agonist.

15. The method of claim 13, wherein the ligand is selected from a group consisting of: Elacridar and Ro 48-8071.

16. The method of claim 13, wherein the ligand is an anti-TMEM97 antibody.

17. A method of treating a subject having a neurological condition, the method comprising administering to the subject a microRNA, siRNA, or antisense RNA that targets TMEM97 expression in an amount and for a duration sufficient to treat the neurological condition.

18. The method of any one of claims 13 to 17, wherein the neurological condition is selected from a group consisting of: conditions requiring neuroprotection, stroke, anxiety, depression, Alzheimer's disease, frontotemporal dementia, Lewy Body dementia, Pick's disease, Huntington's disease, pain, Parkinson's disease, multiple sclerosis, microglia inflammation, schizophrenia, addiction, and brain injury (e.g., concussion or traumatic brain injury).

19. A method of treating cancer, the method comprising administering to the subject a sigma-2 receptor ligand or TMEM97 ligand in an amount and for a duration sufficient to treat the cancer.

20. The method of claim 19, wherein the cancer is squamous cell carcinoma, glioma, colorectal cancer, gastric cancer, epithelial ovarian cancer, ovarian cancer, non-small-cell lung cancer, pancreatic cancer, melanoma, or breast cancer (e.g., triple negative breast cancer), or a multi-drug resistant (MDR) variety of any of the foregoing (e.g., MDR ovarian cancer).

21. The method of claim 19 or 20, wherein the ligand is selected from the group consisting of: compounds of any one of formula I, II, III, IV, V, VI, VII, VIII, IX, X, XI, XII, XIII, XIV, XV, XVI, and XVII.

22. The method of one of claims 1 to 12 and 19 to 21, wherein the sigma-2 receptor ligand is capable of binding a sigma 1 receptor.

23. The method of claim 22, wherein the sigma-2 receptor ligand binds sigma-2 receptor with at least five-fold greater affinity compared to the binding affinity for sigma 1 receptor.

24. The method of one of claims 13 to 21, wherein the TMEM97 ligand is capable of binding a sigma 1 receptor.

25. The method of claim 24, wherein the TMEM97 ligand binds TMEM97 with at least five-fold greater affinity compared to the binding affinity for sigma 1 receptor.

26. A method of treating a subject having a sterol homeostasis disease, the method comprising administering to the subject a composition capable of binding sigma-2 receptor and Sigma 1 receptor, in an amount and for a duration sufficient to treat the sterol homeostasis disease.

27. A method of treating a subject having a neurological condition, the method comprising administering to the subject a composition capable of binding TMEM97 and Sigma 1 receptor, in an amount and for a duration sufficient to treat the neurological condition.

28. A method of treating cancer, the method comprising administering to the subject a subject a composition capable of binding TMEM97 and Sigma 1 receptor, in an amount and for a duration sufficient to treat the cancer.
